SEQUENCE LISTING (63 sequences)

SEQ ID NO: 1 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 3 - protein, 178 aa, Homo sapiens
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 4 - protein, 140 aa, Artificial Sequence
  Engineered TCR alpha constant region
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 5 - protein, 176 aa, Artificial Sequence
  Engineered TCR beta constant region 1
DLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 6 - protein, 178 aa, Artificial Sequence
  Engineered TCR beta constant region 2
DLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 7 - protein, 251 aa, Artificial Sequence
  Engineered MART-1 TCR alpha chain
MKEVEQNSGPLSVPEGAIASLNCTYSYRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 8 - protein, 290 aa, Artificial Sequence
  Engineered MART-1 TCR beta chain
MIAGITQAPTSQILAAGRRMTLRCTQDMRHNAMYWYRQDLGLGLRLIHYSNTAGTTGKGEVPDGYSVSRANTDDFPLTLASAVPSQTSVYFCASSWSFGTEAFFGQGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 9 - protein, 268 aa, Artificial Sequence
  Engineered MART-1 TCR alpha chain
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 10 - protein, 309 aa, Artificial Sequene
  Engineered MART-1 TCR beta chain
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEVGVGQPQHFGDGTRLSILEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 11 - protein, 270 aa, Artificial Sequence
  Engineered WT-1 TCR alpha chain
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAATEDYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 12 - protein, 310 aa, Artificial Sequence
  Engineered WT-1 TCR beta chain
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPGALYEQYFGPGTRLTVTEDLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 13 - protein, 272 aa, Artificial Sequence
  Engineered HPV16 E6 TCR alpha chain
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHPSSNSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 14 - protein, 314 aa, Artificial Sequence
  Engineered HPV16 E6 TCR beta chain
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSSQTGARTDTQYFGPGTRLTVLEDLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 15 - protein, 274 aa, Artificial Sequence
  Engineered NY-ESO-1 TCR alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 16 - protein, 311 aa, Artificial Sequence
  Engineered NY-ESO-1 TCR beta chain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 17 - protein, 274 aa, Artificial Sequence
  Engineered NY-ESO-1 TCR alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 18 - protein, 311 aa, Artificial Sequence
  Engineered NY-ESO-1 TCR beta chain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 19 - protein, 276 aa, Artificial Sequence
  Engineered NY-ESO-1 TCR alpha chain
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 20 - protein, 311 aa, Artificial Sequence
  Engineered NY-ESO-1 TCR beta chain
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 21 - protein, 273 aa, Artificial Sequence
  Engineered NY-ESO-1 TCR alpha chain
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALSANQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 22 - protein, 307 aa, Artificial Sequence
  Engineered NY-ESO-1 TCR beta chain
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSASVATETQYFGPGTRLLVLEDLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 23 - protein, 271 aa, Artificial Sequence
  Engineered HPV16 E7 TCR alpha chain
MKSLRVLLVILWLQLSWVWSQGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPDIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 24 - protein, 314 aa, Artificial Sequence
  Engineered HPV16 E7 TCR beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 25 - protein, 273 aa, Artificial Sequence
  Engineered GP100 TCR alpha chain
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAASLIQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 26 - protein, 310 aa, Artificial Sequence
  Engineered GP100 TCR beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGGNEQFFGPGTRLTVLEDLKNVFPPKVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 27 - protein, 5 aa, Artificial Sequence
  amino acid linker sequence
DGGGS

SEQ ID NO: 28 - protein, 5 aa, Artificial Sequence
  amino acid linker sequence
TGEKP

SEQ ID NO: 29 - protein, 4 aa, Artificial Sequence
  amino acid linker sequence
GGRR

SEQ ID NO: 30 - protein, 5 aa, Artificial Sequence
  amino acid linker sequence
GGGGS

SEQ ID NO: 31 - protein, 14 aa, Artificial Sequence
  amino acid linker sequence
EGKSSGSGSESKVD

SEQ ID NO: 32 - protein, 18 aa, Artificial Sequence
  amino acid linker sequence
KESGSVSSEQLAQFRSLD

SEQ ID NO: 33 - protein, 8 aa, Artificial Sequence
  amino acid linker sequence
GGRRGGGS

SEQ ID NO: 34 - protein, 9 aa, Artificial Sequence
  amino acid linker sequence
LRQRDGERP

SEQ ID NO: 35 - protein, 12 aa, Artificial Sequence
  amino acid linker sequence
LRQKDGGGSERP

SEQ ID NO: 36 - protein, 16 aa, Artificial Sequence
  amino acid linker sequence
LRQKDGGGSGGGSERP

SEQ ID NO: 37 - protein, 18 aa, Artificial Sequence
  amino acid linker sequence
GSTSGSGKPGSGEGSTKG

SEQ ID NO: 38 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 39 - protein, 7 aa, Artificial Sequence
  Cleavage sequence by TEV protease
ENLYFQG

SEQ ID NO: 40 - protein, 7 aa, Artificial Sequence
  Cleavage sequence by TEV protease
ENLYFQS

SEQ ID NO: 41 - protein, 22 aa, Artificial Sequence
  Self-cleaving polypeptide comprising 2A site
GSGATNFSLLKQAGDVEENPGP

SEQ ID NO: 42 - protein, 19 aa, Artificial Sequence
  Self-cleaving polypeptide comprising 2A site
ATNFSLLKQAGDVEENPGP

SEQ ID NO: 43 - protein, 14 aa, Artificial Sequence
  Self-cleaving polypeptide comprising 2A site
LLKQAGDVEENPGP

SEQ ID NO: 44 - protein, 21 aa, Artificial Sequence
  Self-cleaving polypeptide comprising 2A site
GSGEGRGSLLTCGDVEENPGP

SEQ ID NO: 45 - protein, 18 aa, Artificial Sequence
  Self-cleaving polypeptide comprising 2A site
EGRGSLLTCGDVEENPGP

SEQ ID NO: 46 - protein, 13 aa, Artificial Sequence
  Self-cleaving polypeptide comprising 2A site
LLTCGDVEENPGP

SEQ ID NO: 47 - protein, 23 aa, Artificial Sequence
  Self-cleaving polypeptide comprising 2A site
GSGQCTNYALLKLAGDVESNPGP

SEQ ID NO: 48 - protein, 20 aa, Artificial Sequence
  Self-cleaving polypeptide comprising 2A site
QCTNYALLKLAGDVESNPGP

SEQ ID NO: 49 - protein, 14 aa, Artificial Sequence
  Self-cleaving polypeptide comprising 2A site
LLKLAGDVESNPGP

SEQ ID NO: 50 - protein, 25 aa, Artificial Sequence
  Self-cleaving polypeptide comprising 2A site
GSGVKQTLNFDLLKLAGDVESNPGP

SEQ ID NO: 51 - protein, 22 aa, Artificial Sequence
  Self-cleaving polypeptide comprising 2A site
VKQTLNFDLLKLAGDVESNPGP

SEQ ID NO: 52 - protein, 14 aa, Artificial Sequence
  Self-cleaving polypeptide comprising 2A site
LLKLAGDVESNPGP

SEQ ID NO: 53 - protein, 19 aa, Artificial Sequence
  Self-cleaving polypeptide comprising 2A site
LLNFDLLKLAGDVESNPGP

SEQ ID NO: 54 - protein, 19 aa, Artificial Sequence
  Self-cleaving polypeptide comprising 2A site
TLNFDLLKLAGDVESNPGP

SEQ ID NO: 55 - protein, 14 aa, Artificial Sequence
  Self-cleaving polypeptide comprising 2A site
LLKLAGDVESNPGP

SEQ ID NO: 56 - protein, 17 aa, Artificial Sequence
  Self-cleaving polypeptide comprising 2A site
NFDLLKLAGDVESNPGP

SEQ ID NO: 57 - protein, 20 aa, Artificial Sequence
  Self-cleaving polypeptide comprising 2A site
QLLNFDLLKLAGDVESNPGP

SEQ ID NO: 58 - protein, 24 aa, Artificial Sequence
  Self-cleaving polypeptide comprising 2A site
APVKQTLNFDLLKLAGDVESNPGP

SEQ ID NO: 59 - protein, 40 aa, Artificial Sequence
  Self-cleaving polypeptide comprising 2A site
VTELLYRMKRAETYCPRPLLAIHPTEARHKQKIVAPVKQT

SEQ ID NO: 60 - protein, 18 aa, Artificial Sequence
  Self-cleaving polypeptide comprising 2A site
LNFDLLKLAGDVESNPGP

SEQ ID NO: 61 - protein, 40 aa, Artificial Sequence
  Self-cleaving polypeptide comprising 2A site
LLAIHPTEARHKQKIVAPVKQTLNFDLLKLAGDVESNPGP

SEQ ID NO: 62 - protein, 33 aa, Artificial Sequence
  Self-cleaving polypeptide comprising 2A site
EARHKQKIVAPVKQTLNFDLLKLAGDVESNPGP

SEQ ID NO: 63 - DNA, 10 bp, Artificial Sequence
  Consensus Kozak sequence
gccrccatgg

(19) United States
(12) Patent Application Publication
(10) Pub. No.: US 2023/0124640 A1
(43) Pub. Date: Apr. 20, 2023

(54) T CELL RECEPTORS

(71) Applicant: 2seventy bio, Inc., Cambridge, MA (US)

(72) Inventor: JASDEEP MANN, LAKE FOREST PARK, WA (US)

(73) Assignee: 2seventy bio, Inc., Cambridge, MA (US)

(21) Appl. No.: 17/914,483

(22) PCT Filed: Mar. 26, 2021
(86) PCT No.: PCT/US2021/024370

§ 371 Date: Sep. 26, 2022

Related U.S. Application Data
(60) Provisional application No. 63/000,800, filed on Mar. 27, 2020.

(51) Int. Cl. C07K 14/725; C12N 15/86; A61P 35/00

(52) U.S. Cl. CPC C07K 14/7051; C12N 15/86; A61P 35/00; C07K 2319/03

(57) ABSTRACT

The present disclosure provides improved T cell receptors, polynucleotides, polypeptides, vectors, cells, and methods f using the same.

### CROSS REFERENCE TO RELATED APPLICATIONS

This application claims the benefit under 35 U.S.C. § 119(e) of U.S. Provisional Application No. 63/000,800, filed Mar. 27, 2020, which is incorporated by reference herein in its entirety.

### STATEMENT REGARDING SEQUENCE LISTING

The Sequence Listing associated with this application is provided in text format in lieu of a paper copy and is hereby incorporated by reference into the specification. The name of the text file containing the Sequence Listing is BLUE-130_PC_ST25.txt. The text file is 70 KB, created on Mar. 25, 2021, and is being submitted electronically via EFS-Web, concurrent with the filing of the specification.

### BACKGROUND

### Technical Field

The present invention relates to T cell receptors (TCRs) engineered to improve expression and functional avidity. More particularly, the present invention relates to TCRs with amino acid substitutions that improve expression and functional avidity, nucleotides encoding the same, vectors, cells, compositions, medicaments, and methods of using the same.

### Description of the Related Art

Technological advancements in cancer diagnosis and treatment are still out-paced by the poor prognoses that many cancer patients face.

Adoptive cell therapy (ACT) represents a promising approach for the treatment of malignant tumors and virus infections. Adoptive transfer of T lymphocytes genetically modified with antigen-specific T cell receptors (TCR) is an attempt to harness and amplify the tumor-eradicating capacity of a patient's own T cells to eradicate tumors without damaging healthy tissue. In theory, the T cells of the immune system are capable of recognizing protein patterns specific for tumor cells and to mediate their destruction through a variety of effector mechanisms. However, this approach is not new to the field of tumor immunology, and many drawbacks have prevented the widespread use of adoptive T cell therapy for treating cancer and other diseases. A significant obstacle facing TCR gene therapy is TCR mispairing. TCR mispairing is the incorrect pairing between an introduced TCR α or β chain and an endogenous TCR β or α chain, which results in diluted surface expression of the therapeutic αβ TCR and the potential to generate T cells with unknown specificity and toxicity. Another significant limitation of TCR gene therapy is the unpredictable expression of TCRs, which can lead to TCR instability and decreased functional avidity.

### BRIEF SUMMARY

The present disclosure generally relates, in part, to isolated T cell receptors that have been modified (engineered) to increase expression, stability and functional avidity, polynucleotide, compositions, medicaments and uses thereof.

In various embodiments, an isolated T cell receptor (TCR) is provided comprising a minimally murinized TCRα chain and a minimally murinized TCRβ chain, and wherein the TCRα chain transmembrane domain comprises hydrophobic amino acid substitutions.

In various embodiments, an isolated T cell receptor (TCR) is provided comprising a minimally murinized TCRα chain and a minimally murinized TCRβ chain, and wherein the TCRα chain transmembrane domain comprises hydrophobic amino acid substitutions, wherein the TCR does not bind MAGEA4.

In particular embodiments, an isolated T cell receptor (TCR) comprises: a TCRα chain that comprises a constant domain comprising minimal murinization amino acid substitutions at positions 90, 91, 92, and 93, and hydrophobic amino acid substitutions at positions 115, 118, and 119; and a TCRβ chain that comprises a constant domain comprising minimal murinization amino acid substitutions at positions 18, 22, 133, 136, and 139.

In certain embodiments, an isolated T cell receptor (TCR) comprises: a TCRα chain that comprises a constant domain comprising the amino acid substitutions, P90S, E91D, S92V, S93P, S115L, G118V, and F119L; and a TCRβ chain that comprises a constant domain comprising the amino acid substitutions E18K, S22A, F133I, E/V136A, and Q139H. In particular embodiments, an isolated T cell receptor (TCR) comprises: a TCRα chain that comprises a constant domain comprising at least 4 minimal murinization amino acid substitutions and at least 3 hydrophobic amino acid substitutions in the TCRα chain transmembrane domain, wherein the TCRα chain constant domain comprises an amino acid sequence that is at least 95%, 96%, 97%, 98%, or 99% identical to the amino acid sequence set forth in SEQ ID NO: 4; and a TCRβ chain that comprises a constant domain comprising at least 5 minimal murinization amino acid substitutions, wherein the TCRβ chain constant domain comprises an amino acid sequence that is at least 95%, 96%, 97%, 98%, or 99% identical to the amino acid sequence set forth in SEQ ID NO: 5 or SEQ ID NO: 6.

In some embodiments, an isolated TCR comprises a TCRα chain that comprises a constant domain comprising minimal murinization amino acid substitutions at positions 90, 91, 92, and 93, and hydrophobic amino acid substitutions at positions 115, 118, and 119 of the constant region, wherein the amino acid sequence of the TCRα constant region is at least 95%, 96%, 97%, 98%, or 99% identical to the amino acid sequence set forth in SEQ ID NO: 4; and a TCRβ chain that comprises a constant region comprising minimal murinization amino acid substitutions at positions 18, 22, 133, 136, and 139, wherein the amino acid sequence of the TCRβ constant region is at least 95%, 96%, 97%, 98%, or 99% identical to the amino acid sequence set forth in SEQ ID NO: 5 or SEQ ID NO: 6.

In particular embodiments, an isolated TCR comprises a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4; and a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6.

In particular embodiments, an isolated T cell receptor (TCR) comprises: a TCRα chain comprising a constant domain comprising the amino acid sequence set forth in SEQ ID NO: 4; and a TCRβ chain comprising a constant domain comprising the amino acid sequence set forth in SEQ ID NO: 5 or SEQ ID NO: 6.

In particular embodiments, the TCR binds a target antigen selected from the group consisting of: a-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), Latent membrane protein 2 (LMP2), Melanoma antigen family A, 1 (MAGE-A1), MAGE-A2, MAGE-A3, MAGE-A6, MAGE-A10, MAGE-Al2, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2).

In further embodiments, the TCR expression and avidity is increased compared to a TCR that comprises a minimally murinized TCRα chain and a minimally murinized TCRβ chain but wherein the TCRα chain transmembrane domain does not comprise hydrophobic amino acid substitutions.

In additional embodiments, the TCR expression and avidity is increased compared to a TCR that does not comprise a minimally murinized TCRα chain and a minimally murinized TCRβ chain but wherein the TCRα chain transmembrane domain comprises hydrophobic amino acid substitutions. In particular preferred embodiments, an isolated TCR contemplated herein does not bind MAGEA4.

In particular embodiments, a fusion protein is provided comprising a TCR a chain and a TCR β chain contemplated herein.

In particular embodiments, a fusion protein comprises a minimally murinized TCRα chain wherein the TCRα chain transmembrane domain comprises hydrophobic amino acid substitutions; a polypeptide cleavage signal; and a minimally murinized TCRβ chain.

In certain embodiments, a fusion protein comprises: a TCRα chain that comprises a constant domain comprising minimal murinization amino acid substitutions at positions 90, 91, 92, and 93, and hydrophobic amino acid substitutions at positions 115, 118, and 119; a polypeptide cleavage signal; and a TCRβ chain that comprises a constant domain comprising minimal murinization amino acid substitutions at positions 18, 22, 133, 136, and 139.

In particular embodiments, a fusion protein comprises: a TCRα chain that comprises a constant domain comprising the amino acid substitutions, P90S, E91D, S92V, S93P, S115L, G118V, and F119L; a polypeptide cleavage signal; and a TCRβ chain that comprises a constant domain comprising the amino acid substitutions E18K, S22A, F133I, E/V136A, and Q139H.

In particular embodiments, a fusion protein comprises: a TCRα chain that comprises a constant domain comprising at least 4 minimal murinization amino acid substitutions and at least 3 hydrophobic amino acid substitutions in the TCRα chain transmembrane domain, wherein the TCRα chain constant domain comprises an amino acid sequence that is at least 95%, 96%, 97%, 98%, or 99% identical to the amino acid sequence set forth in SEQ ID NO: 4; a polypeptide cleavage signal; and a TCRβ chain that comprises a constant domain comprising at least 5 minimal murinization amino acid substitutions, wherein the TCRβ chain constant domain comprises an amino acid sequence that is at least 95%, 96%, 97%, 98%, or 99% identical to the amino acid sequence set forth in SEQ ID NO: 5 or SEQ ID NO: 6.

In particular embodiments, a fusion protein comprises: a TCRα chain that comprises a constant domain comprising minimal murinization amino acid substitutions at positions 90, 91, 92, and 93, and hydrophobic amino acid substitutions at positions 115, 118, and 119 of the constant region, wherein the amino acid sequence of the TCRα constant region is at least 95%, 96%, 97%, 98%, or 99% identical to the amino acid sequence set forth in SEQ ID NO: 4; a polypeptide cleavage signal; and a TCRβ chain that comprises a constant region comprising minimal murinization amino acid substitutions at positions 18, 22, 133, 136, and 139, wherein the amino acid sequence of the TCRβ constant region is at least 95%, 96%, 97%, 98%, or 99% identical to the amino acid sequence set forth in SEQ ID NO: 5 or SEQ ID NO: 6.

In particular embodiments, a fusion protein comprises: a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4; a polypeptide cleavage signal; and a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6.

In particular embodiments, a fusion protein comprises: a TCRα chain comprising a constant domain comprising the amino acid sequence set forth in SEQ ID NO: 4; a polypeptide cleavage signal; and a TCRβ chain comprising a constant domain comprising the amino acid sequence set forth in SEQ ID NO: 5 or SEQ ID NO: 6.

In some embodiments, the polypeptide cleavage signal is a viral self-cleaving peptide or ribosomal skipping sequence.

In certain embodiments, the polypeptide cleavage signal is a viral 2A peptide.

In further embodiments, the polypeptide cleavage signal is an aphthovirus 2A peptide, a potyvirus 2A peptide, or a cardiovirus 2A peptide.

In additional embodiments, the polypeptide cleavage signal is a viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

In particular preferred embodiments, a fusion protein contemplated herein does not bind MAGEA4.

In other embodiments, a nucleic acid encodes a TCR or a fusion protein contemplated herein.

In some embodiments, a nucleic acid comprises a first polynucleotide encoding a minimally murinized TCRα chain wherein the TCRα chain transmembrane domain comprises hydrophobic amino acid substitutions; an internal ribosomal entry site (IRES); and a second polynucleotide encoding a minimally murinized TCRβ chain.

In particular embodiments, a nucleic acid comprises: a first polynucleotide encoding a TCRα chain that comprises a constant domain comprising minimal murinization amino acid substitutions at positions 90, 91, 92, and 93, and hydrophobic amino acid substitutions at positions 115, 118, and 119; an IRES; and a second polynucleotide encoding a TCRβ chain that comprises a constant domain comprising minimal murinization amino acid substitutions at positions 18, 22, 133, 136, and 139.

In certain embodiments, a nucleic acid comprises: a first polynucleotide encoding a TCRα chain that comprises a constant domain comprising the amino acid substitutions, P90S, E91D, S92V, S93P, S115L, G118V, and F119L; an IRES; and a second polynucleotide encoding a TCRβ chain that comprises a constant domain comprising the amino acid substitutions E18K, S22A, F133I, E/V136A, and Q139H.

In some embodiments, a nucleic acid comprises: a first polynucleotide encoding a TCRα chain that comprises a constant domain comprising at least 4 minimal murinization amino acid substitutions and at least 3 hydrophobic amino acid substitutions in the TCRα chain transmembrane domain, wherein the TCRα chain constant domain comprises an amino acid sequence that is at least 95%, 96%, 97%, 98%, or 99% identical to the amino acid sequence set forth in SEQ ID NO: 4; an IRES; and a second polynucleotide encoding a TCRβ chain that comprises a constant domain comprising at least 5 minimal murinization amino acid substitutions, wherein the TCRβ chain constant domain comprises an amino acid sequence that is at least 95%, 96%, 97%, 98%, or 99% identical to the amino acid sequence set forth in SEQ ID NO: 5 or SEQ ID NO: 6.

In preferred embodiments, a nucleic acid comprises: a first polynucleotide encoding a TCRα chain that comprises a constant domain comprising minimal murinization amino acid substitutions at positions 90, 91, 92, and 93, and hydrophobic amino acid substitutions at positions 115, 118, and 119 of the constant region, wherein the amino acid sequence of the TCRα constant region is at least 95%, 96%, 97%, 98%, or 99% identical to the amino acid sequence set forth in SEQ ID NO: 4; an IRES; and a second polynucleotide encoding a TCRβ chain that comprises a constant region comprising minimal murinization amino acid substitutions at positions 18, 22, 133, 136, and 139, wherein the amino acid sequence of the TCRβ constant region is at least 95%, 96%, 97%, 98%, or 99% identical to the amino acid sequence set forth in SEQ ID NO: 5 or SEQ ID NO: 6.

In particular embodiments, a nucleic acid comprises: a first polynucleotide encoding a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4; an IRES; and a second polynucleotide encoding a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6.

In certain embodiments, a nucleic acid comprises: a first polynucleotide encoding a TCRα chain comprising a constant domain comprising the amino acid sequence set forth in SEQ ID NO: 4; an IRES; and a second polynucleotide encoding a TCRβ chain comprising a constant domain comprising the amino acid sequence set forth in SEQ ID NO: 5 or SEQ ID NO: 6.

In particular preferred embodiments, nucleic acids contemplated herein do not encode isolated TCRs or fusion proteins that bind MAGEA4.

In some embodiments, a vector comprises a nucleic acid encoding a TCR or a fusion protein contemplated herein.

In particular embodiments, a vector comprises a nucleic acid contemplated herein.

In some embodiments, the vector is an expression vector.

In other embodiments, the vector is a retroviral vector or a lentiviral vector.

In particular embodiments, a cell is modified to express a TCR contemplated herein.

In certain embodiments, a cell is modified to express a fusion protein contemplated.

In particular embodiments, a cell is modified to express a nucleic acid contemplated herein.

In particular embodiments, a cell comprises a vector contemplated herein.

In certain embodiments, the cell is an immune effector cell.

In further embodiments, the cell is an immune effector cell selected from the group consisting of: a T cell, a natural killer (NK) cell, or a natural killer T (NKT) cell.

In various embodiments, a composition comprises a TCR, a fusion protein, a nucleic acid, a vector, or a cell contemplated herein.

In various embodiments, a pharmaceutical composition comprising a pharmaceutically acceptable carrier and a TCR, a fusion protein, a nucleic acid, a vector, or a cell contemplated herein.

A TCR, a fusion protein, a nucleic acid, a vector, a cell, a composition, or a pharmaceutical composition contemplated herein for use as a medicament.

A TCR, a fusion protein, a nucleic acid, a vector, a cell, a composition, or a pharmaceutical composition contemplated herein for use in the treatment of cancer, wherein the cancer is preferably a hematological cancer or a solid tumor, more preferably wherein the cancer is selected from the group consisting of sarcoma, prostate cancer, uterine cancer, thyroid cancer, testicular cancer, renal cancer, pancreatic cancer, ovarian cancer, esophageal cancer, non-small-cell lung cancer, non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia, most preferably wherein the cancer is selected from the group consisting of NSCLC, SCLC, breast, ovarian or colorectal cancer, sarcoma or osteosarcoma.

### BRIEF DESCRIPTION OF SEVERAL VIEWS OF THE DRAWINGS

FIG. 1 shows the effect of various amino acid substitutions on the MAGEA4 TCR expression. Donor cells (n=2) were transduced with lentiviral constructs encoding: TCR^{TM} (hydrophobic mutations in transmembrane domain), TCR^{MM} (minimal murine mutations), TCR^{TM/MM} (both set of mutations) or TCR^{WT} (human TCR) and cultured for 10 days. Untransduced (UTD) T cells were used as a control. Expression was validated by labelling with MAGEA4 peptide (pentamer configuration) on day 10.

FIG. 2A shows increased TCR expression in T cells transduced with the lentiviral vector encoding TCR^{TM/MM} compared to T cells transduced with the lentiviral vector encoding TCR^{WT} under two different transduction (Tdxn) conditions (left panel).

FIG. 2B shows comparable transduction efficiencies of donor cells (n=2) with the lentiviral TCR constructs, TCR^{TM/MM} and TCR^{WT} under two different Tdxn conditions (right panel).

FIG. 3A shows TCR mispairing in a T cell expressing a TCR^{WT}. Pairing is expressed as percentage positive cells detected by v-beta staining and tetramer antigen staining. Specific TCR pairing was indicated where the percentage positive cells detected by v-beta staining and tetramer antigen staining were equal.

FIG. 3B shows TCR mispairing in a T cell expressing a TCR^{TM/MM} (right panel). Pairing is expressed as percentage positive cells detected by v-beta staining and tetramer antigen staining. Specific TCR pairing was indicated where the percentage positive cells detected by v-beta staining and tetramer antigen staining were equal.

FIG. 4A shows the IFNγ production from donor T cells transduced with lentiviral vectors encoding TCR^{WT} and TCR^{TM/MM}. UTD T cells, TCR^{WT}, or TCR^{TM/MM} T-cells were co-cultured with MAGEA4 expressing tumor cells at an E:T of 1:1 and IFNγ expression was measured 24 hours later (left panel).

FIG. 4B shows the cytotoxicity of donor T cells transduced with lentiviral vectors encoding TCR^{WT} and TCR^{TM/MM}. UTD T cells, TCR^{WT}, or TCR^{TM/MM} T-cells were co-cultured with MAGEA4 expressing tumor cells at an E:T of 1:1 and cytotoxicity was measured over a 3 day period (right panel).

FIGS. 5A and 5B show the effects of various mutations on the NY-ESO-1 TCR expression. Donor cells (n=2) were transduced with lentiviral constructs encoding: TCR^{WT} (human TCR), TCR^{MM} (minimal murine mutations), or TCR^{TM/MM} (hydrophobic mutations in transmembrane domain and minimal murine mutations) and cultured for 10 days. Untransduced (UTD) T cells were used as a control. FIG. 5A shows NY-ESO-1 TCR expression validated by labelling with NY-ESO peptide (pentamer configuration) on day 10. FIG. 5B shows Mean Fluorescence Intensity (MFI) of NY-ESO-1 TCR expression for each donor.

FIG. 6 shows NY-ESO-1 TCR mispairing in UTD T cells, TCR^{WT} T cells and TCR^{TM/MM} T cells. Pairing is expressed as percentage positive cells detected by v-beta staining and tetramer antigen staining.

### BRIEF DESCRIPTION OF THE SEQUENCE IDENTIFIERS

SEQ ID NO: 1 sets forth the amino acid sequence of human TCRα constant region.

SEQ ID NO: 2 sets forth the amino acid sequence of human TCRβ constant region 1.

SEQ ID NO: 3 sets forth the amino acid sequence of human TCRβ constant region 2.

SEQ ID NO: 4 sets forth the amino acid sequence of human TCRα constant region comprising minimal murine amino acid substitutions and hydrophobic amino acid substitutions in transmembrane domain.

SEQ ID NO: 5 sets forth the amino acid sequence of human TCRβ constant region 1 comprising minimal murine amino acid substitutions.

SEQ ID NO: 6 sets forth the amino acid sequence of human TCRβ constant region 2 comprising minimal murine amino acid substitutions.

SEQ ID NO: 7 sets forth the amino acid sequence of a human MART-1 TCRα chain comprising a constant region with minimal murine amino acid substitutions and hydrophobic amino acid substitutions in transmembrane domain.

SEQ ID NO: 8 sets forth the amino acid sequence of human MART-1 TCRβ chain comprising a constant region with minimal murine amino acid substitutions.

SEQ ID NO: 9 sets forth the amino acid sequence of a human MART-1 TCRα chain comprising a constant region with minimal murine amino acid substitutions and hydrophobic amino acid substitutions in transmembrane domain.

SEQ ID NO: 10 sets forth the amino acid sequence of human MART-1 TCRβ chain comprising a constant region with minimal murine amino acid substitutions.

SEQ ID NO: 11 sets forth the amino acid sequence of a human WT-1 TCRα chain comprising a constant region with minimal murine amino acid substitutions and hydrophobic amino acid substitutions in transmembrane domain.

SEQ ID NO: 12 sets forth the amino acid sequence of human WT-1 TCRβ chain comprising a constant region with minimal murine amino acid substitutions.

SEQ ID NO: 13 sets forth the amino acid sequence of a human HPV16 E6 TCRa chain comprising a constant region with minimal murine amino acid substitutions and hydrophobic amino acid substitutions in transmembrane domain.

SEQ ID NO: 14 sets forth the amino acid sequence of human HPV16 E6 TCRβ chain comprising a constant region with minimal murine amino acid substitutions.

SEQ ID NO: 15 sets forth the amino acid sequence of a human NY-ESO-1 TCRα chain comprising a constant region with minimal murine amino acid substitutions and hydrophobic amino acid substitutions in transmembrane domain.

SEQ ID NO: 16 sets forth the amino acid sequence of human NY-ESO-1 TCRβ chain comprising a constant region with minimal murine amino acid substitutions.

SEQ ID NO: 17 sets forth the amino acid sequence of a human NY-ESO-1 TCRα chain comprising a constant region with minimal murine amino acid substitutions and hydrophobic amino acid substitutions in transmembrane domain.

SEQ ID NO: 18 sets forth the amino acid sequence of human NY-ESO-1 TCRβ chain comprising a constant region with minimal murine amino acid substitutions.

SEQ ID NO: 19 sets forth the amino acid sequence of a human NY-ESO-1 TCRα chain comprising a constant region with minimal murine amino acid substitutions and hydrophobic amino acid substitutions in transmembrane domain.

SEQ ID NO: 20 sets forth the amino acid sequence of human NY-ESO-1 TCRβ chain comprising a constant region with minimal murine amino acid substitutions.

SEQ ID NO: 21 sets forth the amino acid sequence of a human NY-ESO-1 TCRα chain comprising a constant region with minimal murine amino acid substitutions and hydrophobic amino acid substitutions in transmembrane domain.

SEQ ID NO: 22 sets forth the amino acid sequence of human NY-ESO-1 TCRβ chain comprising a constant region with minimal murine amino acid substitutions.

SEQ ID NO: 23 sets forth the amino acid sequence of a human HPV16 E7 TCRα chain comprising a constant region with minimal murine amino acid substitutions and hydrophobic amino acid substitutions in transmembrane domain.

SEQ ID NO: 24 sets forth the amino acid sequence of human HPV16 E7 TCRβ chain comprising a constant region with minimal murine amino acid substitutions.

SEQ ID NO: 25 sets forth the amino acid sequence of a human GP100 TCRα chain comprising a constant region with minimal murine amino acid substitutions and hydrophobic amino acid substitutions in transmembrane domain.

SEQ ID NO: 26 sets forth the amino acid sequence of human GP100 TCRβ chain comprising a constant region with minimal murine amino acid substitutions.

SEQ ID NOs: 27-37 set forth the amino acid sequences of various linkers. SEQ ID NOs: 38-62 set forth the amino acid sequences of protease cleavage sites and self-cleaving polypeptide cleavage sites.

SEQ ID NO: 63 sets for the polynucleotide sequence of a consensus Kozak sequence. Throughout the disclosure, reference is made to amino acid positions with the TCRα and TCRβ constant regions. The amino acid positions are numbered with reference to SEQ ID NOs: 1 and 4 for TCRα and SEQ ID NOs: 2, 3, 5, and 6 for TCRβ.

In the foregoing sequences, X, if present, refers to any amino acid or the absence of an amino acid.

### DETAILED DESCRIPTION

### A. Overview

The present disclosure generally relates to, in part, T cell receptors that have been modified to increase expression, stability and functional avidity. TCR avidity is determined by a TCR's affinity for its target peptide and TCR expression. TCR affinities for target peptides are generally in the range of 1 μM-10 ηM. However, if TCR affinity is too high it could lead to either thymic rejection or unwanted off-target activity. TCR avidity can also be enhanced by increasing the number of TCR molecules being expressed on the cell surface, which can potentially be achieved through codon optimization and optimization of chain orientation for balanced expression. TCR stability also can play a role in TCR expression.

Positively charged residues in the TCR transmembrane region may contribute to TCR instability and decreased expression. Although changing the composition of the transmembrane domains may decrease instability and increase expression, the chains are not amenable to drastic changes, since some charged residues are critical for interaction with CD3 complex.

In addition to TCR chain instability, low expression also stems from competition with the endogenous TCR chains. Mispairing of transgenic (exogenous) chains with endogenous chains leads to low TCR expression and decreases TCR functional avidity and potential off-target toxicity. The art has addressed this problem through knockout of the endogenous TCR locus and replacing transgenic constant domains (e.g., human) with constant domains from a different species (e.g., mouse). These strategies suffer from incomplete inactivation of the endogenous TCR locus and an increased risk of immunogenicity due to the presence of the foreign constant domains.

The present inventor(s) have unexpectedly discovered that TCRs engineered with a combination of minimal murine amino acid substitutions and hydrophobic amino acid substitutions in the TCRα transmembrane domain synergistically increases TCR stability, expression, specific pairing and functional avidity. Moreover, the present inventors have surprisingly discovered that engineering the TCR constant domains can imbue many TCRs (both high affinity and low affinity) with the foregoing characteristics; thus, making them a more tractable immunotherapy strategy. In addition, the engineered TCRs contemplated herein offer other advantages over engineered TCR T cells in the art, including a simplified manufacturing process, reduced number of TCR T cells to meet dose, and possibility of further engineering without reducing TCR expression.

In various embodiments, T cell receptors (TCRs) engineered for increased stability, expression, and functional avidity are provided. The TCRs contemplated herein comprise one or more amino acid substitutions to minimally murinize the TCR and one or more amino acid hydrophobic amino acid substitutions in the transmembrane domain. In particular embodiments, the TCRs comprise a TCRα chain with a constant region that has been minimally murinized and that contains hydrophobic amino acid substitutions in the transmembrane domain and a TCRα chain with a constant region that has been minimally murinized.

In particular embodiments, a TCR contemplated herein comprises 1, 2, 3, or 4 amino acid substitutions in a TCRα constant region to minimally murinize the TCRα chain; 1, 2, or 3 hydrophobic amino acid substitutions in a TCRα transmembrane domain; and 1, 2, 3, 4, or 5 amino acid substitutions in a TCRβ constant region to minimally murinize the TCRβ chain. In preferred embodiments, a TCR contemplated herein comprises 4 amino acid substitutions in a TCRα constant region to minimally murinize the TCRα chain; 3 hydrophobic amino acid substitutions in a TCRα transmembrane domain; and 5 amino acid substitutions in a TCRβ constant region to minimally murinize the TCRβ chain.

TCRs contemplated herein typically bind a target antigen presented by a major histocompatibility complex (MHC) molecule. In particular embodiments, TCRs contemplated herein bind a target antigen that is expressed on a cancer cell, i.e., a tumor antigen, including but not limited to tumor associated antigens (TAA) and tumor specific antigens (TSA).

In particular embodiments, one or more polynucleotides encoding an engineered TCR is contemplated. A TCRα chain and a TCRβ chain can be encoded by different polynucleotides, or by a single polynucleotide as a polycistronic protein or as a fusion polypeptide wherein the chains are separated by a polynucleotide encoding a linker polypeptide, optionally a self-cleaving polypeptide. In particular embodiments, a polynucleotide encodes a TCRα chain, a self-cleaving polypeptide, and a TCRβ polypeptide. In other particular embodiments, a polynucleotide encodes a TCRβ chain, a self-cleaving polypeptide, and a TCRα polypeptide.

It is further contemplated that in particular embodiments, TCR polynucleotides are introduced into immune effector cells. Immune effector cells expressing the TCRs completed herein may be formulated as compositions or pharmaceutical compositions and can be used in the manufacture of a medicament for treating cancer and/or in methods of treating cancer.

In preferred embodiments, the TCRs contemplated herein do not bind MAGEA4, including but not limited to primate or human MAGEA4.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir andCC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

An “exogenous” molecule is a molecule that is not normally present in a cell, but that is introduced into a cell by one or more genetic, biochemical or other methods. Exemplary exogenous molecules include, but are not limited to small organic molecules, protein, nucleic acid, carbohydrate, lipid, glycoprotein, lipoprotein, polysaccharide, any modified derivative of the above molecules, or any complex comprising one or more of the above molecules.

Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. Additional endogenous molecules can include proteins.

Additional definitions are set forth throughout this disclosure.

### C. T Cell Receptors

T cell receptors (TCRs) recognize a peptide fragment of a target antigen when it is presented by a major histocompatibility complex (MEC) molecule. There are two different classes of MEC molecules, MEC I and MEC II, that deliver peptides from different cellular compartments to the cell surface. Engagement of the TCR with antigen and MEC results in immune effector cell activation through a series of biochemical events mediated by associated enzymes, co-receptors, and specialized accessory molecules.

A TCR contemplated herein is a heterodimeric complex comprising a TCR alpha (TCRα) chain and a TCR beta (TCRβ) chain. The human TCRα locus is located on chromosome 14 (14q11.2). The mature TCRα chain comprises a variable domain derived from recombination of a variable (V) segment and a joining (J) segment, and a constant (C) domain. The term “variable TCRα region” or “TCRα variable chain” or “TCRα variable domain” refers to the variable region of a TCRα chain. The human TCRβ locus is located on chromosome 7 (7q34). The mature TCRβ chain comprises a variable domain derived from recombination of a variable (V) segment, a diversity (D) segment, and a joining (J) segment, and one of two constant (C) domains. The term “variable TCR β region” or “TCR β variable chain” or “TCRβ variable domain” refers to the variable region of a TCR β chain.

The rearranged V(D)J regions of both the TCRα chain and the TCRβ chain each contain three hypervariable regions known as complementarity determining regions (CDRs). CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MEW molecule. Framework regions (FRs) are positioned between the CDRs. These regions provide the structure of the TCR variable region.

The constant domain or constant region of the TCR chain also contributes to TCR structure and consists of an extracellular domain, a transmembrane domain and a short cytoplasmic domain.

The TCR structure allows the formation of a TCR complex that includes the TCRα chain, the TCRβ chain and accessory molecules CD3γ, CD3δ, CD3ε, and CD3ζ. The signal from the T cell complex is enhanced by simultaneous binding of the MEW molecules by a specific co-receptor. CD4 is the co-receptor for MEW II molecules expressed on helper T cells and CD8 is the co-receptor for MEW I molecules expressed on cytotoxic T cells. The co-receptor not only ensures the specificity of the TCR for an antigen, but also allows prolonged engagement between the antigen presenting cell and the T cell and recruits essential molecules (e.g., LCK) inside the cell involved in the signaling of the activated T lymphocyte.

TCRs contemplated herein can be used to redirect immune effector cells to target cells. The TCRs contemplated herein are engineered to increase TCR stability, TCR expression, specific TCR pairing and functional avidity.

In particular embodiments, the constant domains of the TCRα and TCRβ chains are engineered or modified to increase TCR stability, TCR expression, specific TCR pairing, and functional avidity.

To efficiently enhance correct pairing the engineered TCR sequences and to avoid mispairing with endogenous TCR chains, the engineered TCR sequences are modified to minimally murinize the TCRα and TCRβ constant domains. Murinization of TCRs refers to exchanging the human TCRα and TCRβ constant domains with their murine counterparts. Nine amino acids responsible for the improved expression of murinized TCRs have been identified. “Minimal murinization” offers the advantage of enhancing cell surface expression while, at the same time, reducing the number of “foreign” amino acid residues in the amino acid sequence and, thereby, reducing the risk of immunogenicity. Minimal murinization refers to the substitution of the 1, 2, 3, or 4 amino acids, preferably all 4 amino acids, in the TCRα constant domain and substitution of the 1, 2, 3, 4, or 5 amino acids, preferably all 5 amino acids, in the TCRβ constant domain that are responsible for the improved expression in murinized TCRs. In preferred embodiments, minimal murinization refers to the substitution of the 4 amino acids in the human TCRα constant domain and substitution of the 5 amino acids in the human TCRβ constant domain that are responsible for the improved expression in murinized TCRs.

The engineered or modified TCRs contemplated herein comprise minimally murinized TCRα and TCRβ constant domains and further comprise hydrophobic amino acid substitutions in the TCRα transmembrane domain to increase TCR stability, TCR expression, and functional avidity. The transmembrane domain of the TCR a chain has been shown to contribute to the lack of stability of the whole chain and thereby affecting the formation and surface expression of the whole TCR—CD3 complex. In particular embodiments, substitution of 1, 2, or 3 amino acids, preferably all 3 amino acids, in the TCR a transmembrane domain with hydrophobic amino acids improves TCR stability, expression, and avidity. In preferred embodiments, the TCR a transmembrane domain comprises 3 hydrophobic amino acids substitutions to improve TCR stability, expression, and avidity.

Illustrative examples of hydrophobic amino acids suitable for use in particular embodiments include alanine, (A), valine (V), isoleucine (I), leucine (L), methionine (M), phenylalanine (F), tyrosine (Y), and trypophan (W). In preferred embodiments, hydrophobic amino acids are selected from the group consisting of alanine, (A), valine (V), isoleucine (I), and leucine (L). In more preferred embodiments, hydrophobic amino acids are selected from the group consisting of valine (V), isoleucine (I), and leucine (L). In even more preferred embodiments, the hydrophobic amino acids are valine (V) and leucine (L).

In particular embodiments, an engineered TCR comprises a minimally murinized TCRα chain and a minimally murinized TCRβ chain, wherein the TCRα chain transmembrane domain comprises hydrophobic amino acid substitutions.

In preferred embodiments, an engineered TCR comprises a minimally murinized TCRα chain comprising 4 amino acid substitutions in the TCRα constant region and a minimally murinized TCRβ chain comprising 5 amino acid substitutions in the TCRβ constant region, wherein the TCRα chain transmembrane domain further comprises three hydrophobic amino acid substitutions.

In preferred embodiments, an engineered TCR comprises a TCRα chain that comprises a constant domain comprising minimal murinization amino acid substitutions at positions 90, 91, 92, and 93, and hydrophobic amino acid substitutions at positions 115, 118, and 119 of the constant region; and a TCRβ chain that comprises a constant domain comprising minimal murinization amino acid substitutions at positions 18, 22, 133, 136, and 139.

In preferred embodiments, an engineered TCR comprises a TCRα chain that comprises a constant domain comprising the following minimal murinization amino acid substitutions, P90S, E91D, S92V, and S93P and the following hydrophobic amino acid substitutions in the transmembrane domain of the constant region, S115L, G118V, and F119L; and a TCRβ chain that comprises a constant domain comprising the following minimal murinization amino acid substitutions, E18K, S22A, F133I, E/V136A, and Q139H.

In preferred embodiments, an engineered TCR comprises a TCRα chain that comprises a constant domain comprising minimal murinization amino acid substitutions at positions 90, 91, 92, and 93, and hydrophobic amino acid substitutions at positions 115, 118, and 119 of the constant region, wherein the amino acid sequence of the TCRα constant region is at least 95%, 96%, 97%, 98%, or 99% identical to the amino acid sequence set forth in SEQ ID NO: 4; and a TCRβ chain that comprises a constant region comprising minimal murinization amino acid substitutions at positions 18, 22, 133, 136, and 139, wherein the amino acid sequence of the TCRβ constant region is at least 95%, 96%, 97%, 98%, or 99% identical to the amino acid sequence set forth in SEQ ID NO: 5 or SEQ ID NO: 6.

In particular embodiments, an engineered TCR comprises a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4; and a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6.

In particular preferred embodiments, an engineered TCR comprises a TCRα chain that comprises a constant domain comprising the amino acid sequence set forth in SEQ ID NO: 4; and a TCRβ chain that comprises a constant domain comprising the amino acid sequence set forth in SEQ ID NO: 5 or SEQ ID NO: 6.

In particular embodiments, an engineered TCR comprises variable domains that bind an antigen. In preferred embodiments, the antigen is not MAGEA4.

### D. Target Antigens

The engineered T cell receptors (TCRs) contemplated herein bind a polypeptide antigen presented by a major histocompatibility complex (MHC) class I or MHC class II molecule, preferentially a polypeptide antigen presented by an MHC class I molecule.

“Major histocompatibility complex” (MHC) refers to glycoproteins that deliver peptide antigens to a cell surface. MHC class I molecules are heterodimers having a membrane spanning a chain (with three a domains) and a non-covalently associated (32 microglobulin. MHC class II molecules are composed of two transmembrane glycoproteins, a and (3, both of which span the membrane. Each chain has two domains. MHC class I molecules deliver peptides originating in the cytosol to the cell surface, where a peptide:MHC complex is recognized by CD8⁺ T cells. MEC class II molecules deliver peptides originating in the vesicular system to the cell surface, where they are recognized by CD4⁺ T cells. Human MEC is referred to as human leukocyte antigen (HLA).

“Antigen (Ag),” “target antigen,” and “polypeptide antigen” are used interchangeably in preferred embodiments are collective refer to a naturally processed or synthetically produced portion of an antigenic protein, e.g., a tumor associated antigen (TAA) or tumor specific antigen (TSA), ranging in length from about 7 amino acids to about 15 amino acids, which can form a complex with a MEC (e.g., HLA) molecule forming a target antigen:MEC (e.g., HLA) complex.

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MEC gene that is relevant for antigen presentation) APC and T cells, are well established (see, e.g., Murphy, Janeway's Immunobiology (8th Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MEC molecules, whereas peptides processed in the vesicular system (e.g., bacterial, viral) will generally vary in length from about 10 amino acids to about 25 amino acids and associate with class II MEC molecules.

In particular embodiments, an engineered TCR contemplated herein binds a tumor antigen, e.g., a TAA or TSA. “Tumor associate antigens” or “TAAs” include but are not limited to oncofetal antigens, overexpressed antigens, lineage restricted antigens, and cancer-testis antigens. TAAs are relatively restricted to tumor cells. TAAs have elevated expression levels on tumor cells, but are also expressed at lower levels on healthy cells. “Tumor-specific antigens” or “TSAs” include but are not limited to neoantigens and oncoviral antigens. TSAs are unique to tumor cells. TSAs are expressed in cancer cells and not normal cells.

In particular embodiments, engineered TCRs contemplated herein bind an antigenic portion of a polypeptide selected from the group consisting of: a-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), Latent membrane protein 2 (LMP2), Melanoma antigen family A, 1 (MAGE-A1), MAGE-A2, MAGE-A3, MAGE-A6, MAGE-A10, MAGE-Al2, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (55X4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2).

In particular embodiments, engineered TCRs contemplated herein bind an antigenic portion of a polypeptide selected from the group consisting of: CT-83, MAGE-A3, MART-1, MUC16, NY-ESO-1, PLAC-1, PRAME, SSX2, Survivin, and WT-1

In particular embodiments, engineered TCRs contemplated herein bind an antigenic portion of NY-ESO-1.

### E. Polypeptides

Various polypeptides, fusion polypeptides, and polypeptide variants are contemplated herein, including, but not limited to, TCR polypeptides, TCRα chain polypeptides, TCRβ chain polypeptides, TCR fusion polypeptides, and fragments thereof. In particular embodiments, exemplary polypeptides contemplated herein include polypeptides comprising an amino acid sequence as set forth in any one of SEQ ID NOs: 4-26.

“Polypeptide,” “peptide” and “protein” are used interchangeably, unless specified to the contrary, and according to conventional meaning, i.e., as a sequence of amino acids. Polypeptides are not limited to a specific length, e.g., they may comprise a full-length polypeptide or a polypeptide fragment, and may include one or more post-translational modifications of the polypeptide, for example, glycosylations, acetylations, phosphorylations and the like, as well as other modifications known in the art, both naturally occurring and non-naturally occurring.

An “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances. In particular embodiments, an isolated polypeptide is a synthetic polypeptide, a recombinant polypeptide, or a semi-synthetic polypeptide, or a polypeptide obtained or derived from a recombinant source.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more of the polypeptide sequences contemplated herein. For example, in particular embodiments, it may be desirable to improve the binding affinity, stability, expression, specific pairing, functional avidity and/or other biological properties of a TCR by introducing one or more substitutions, deletions, additions and/or insertions into a TCRα chain and/or TCRβ chain. In particular embodiments, polypeptides include polypeptides having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any of the polypeptide sequences contemplated herein, typically where the variant maintains at least one biological activity of the reference sequence.

Polypeptides include “polypeptide fragments.” Polypeptide fragments refer to a polypeptide, which can be monomeric or multimeric that has an amino-terminal deletion, a carboxyl-terminal deletion, and/or an internal deletion or substitution of a naturally-occurring or recombinantly-produced polypeptide. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long.

As noted above, in particular embodiments, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif, 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In preferred embodiments, fusion polypeptides are contemplated herein. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. In particular embodiments, polypeptides of the fusion protein can be in any order or a specified order.

In particular embodiments, a TCR contemplated herein is expressed as a fusion polypeptide that comprises a TCRα chain, a polypeptide linker, and a TCRβ chain. In some embodiments, a TCR is expressed as a fusion protein that comprises from 5′ to 3′, a TCRα chain, a polypeptide linker, and a TCRβ chain. In some embodiments, a TCR is expressed as a fusion protein that comprises from 5′ to 3′, a TCRβ chain, a polypeptide linker, and a TCRα chain.

A “linker” is an amino acid sequence that connect adjacent domains of a polypeptide or fusion polypeptide. Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers known in the art. Glycine accesses significantly more phi-psi space than even alanine, and is much less restricted than residues with longer side chains (see

Scheraga, Rev. Computational Chem. 11173-142 (1992)). A linker may be 1, 2, 3, 4, 5, 6, 7, 8, 9, 10, 11, 12, 13, 14, 15, 16, 17, 18, 19, 20, 21, 22, 23, 24, 25, or more amino acids long. Other exemplary linkers include, but are not limited to the following amino acid sequences: DGGGS (SEQ ID NO: 27); TGEKP (SEQ ID NO: 28) (see, e.g., Liu et al., PNAS 5525-5530 (1997)); GGRR (SEQ ID NO: 29) (Pomerantz et al. 1995, supra); (GGGGS)ₙ where n =1, 2, 3, 4 or 5 (SEQ ID NO: 30) (Kim et al., PNAS 93, 1156-1160 (1996.); EGKSSGSGSESKVD (SEQ ID NO: 31) (Chaudhary et al., 1990, Proc. Natl. Acad. Sci. U.S.A. 87:1066-1070); KESGSVSSEQLAQFRSLD (SEQ ID NO: 32) (Bird et al., 1988, Science 242:423-426), GGRRGGGS (SEQ ID NO: 33); LRQRDGERP (SEQ ID NO: 34); LRQKDGGGSERP (SEQ ID NO: 35); LRQKD(GGGS)₂ ERP (SEQ ID NO: 36). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In particular embodiments, a linker comprises the amino acid sequence: GSTSGSGKPGSGEGSTKG (SEQ ID NO: 37) (Cooper et al., Blood, 101(4): 1637-1644 (2003)).

In particular embodiments, a fusion polypeptide comprises a minimally murinized TCRα chain, a polypeptide linker, and a minimally murinized TCRβ chain, wherein the TCRα chain transmembrane domain comprises hydrophobic amino acid substitutions. In some embodiments, a fusion protein that comprises from 5′ to 3′, a minimally murinized TCRα chain, a polypeptide linker, and a minimally murinized TCRβ chain, wherein the TCRα chain transmembrane domain comprises hydrophobic amino acid substitutions. In some embodiments, a fusion protein that comprises from 5′ to 3′, a minimally murinized TCRβ chain, a polypeptide linker, and a minimally murinized TCRα chain, wherein the TCRα chain transmembrane domain comprises hydrophobic amino acid substitutions.

In particular embodiments, a fusion polypeptide comprises a minimally murinized TCRα chain comprising 4 amino acid substitutions in the TCRα constant region, a polypeptide linker, and a minimally murinized TCRβ chain comprising 5 amino acid substitutions in the TCRβ constant region, wherein the TCRα chain transmembrane domain further comprises three hydrophobic amino acid substitutions. In some embodiments, a fusion protein comprises from 5′ to 3′, a minimally murinized TCRα chain comprising 4 amino acid substitutions in the TCRα constant region, a polypeptide linker, and a minimally murinized TCRβ chain comprising 5 amino acid substitutions in the TCRβ constant region, wherein the TCRα chain transmembrane domain further comprises three hydrophobic amino acid substitutions. In some embodiments, a fusion protein that comprises from 5′ to 3′, a minimally murinized TCRβ chain comprising 5 amino acid substitutions in the TCRβ constant region, a polypeptide linker, and a minimally murinized TCRα chain comprising 4 amino acid substitutions in the TCRα constant region, wherein the TCRα chain transmembrane domain further comprises three hydrophobic amino acid substitutions.

In particular embodiments, a fusion polypeptide comprises a minimally murinized TCRα chain (e.g., SEQ ID NOs:7, 9, 11, 13, 15, 17, 19, 21, 23, and 25) comprising the following minimal murinization amino acid substitutions, P90S, E91D, S92V, and S93P and the following hydrophobic amino acid substitutions in the transmembrane domain, S115L, G118V, and F119L of the TCRα constant domain, a polypeptide linker, and a TCRβ chain (e.g., SEQ ID NOs:8, 10, 12, 14, 16, 18, 20, 22, 24, and 26) that comprises a constant domain comprising the following minimal murinization amino acid substitutions, E18K, S22A, F133I, E/V136A, and Q139H. In some embodiments, a fusion protein comprises from 5′ to 3′, a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the TCRα constant domain, a polypeptide linker, and a TCRβ chain that comprises a constant domain comprising the following minimal murinization amino acid substitutions, E18K, S22A, F133I, E/V136A, and Q139H. In some embodiments, a fusion protein that comprises from 5′ to 3′, a TCRβ chain that comprises a constant domain comprising the following minimal murinization amino acid substitutions, E18K, S22A, F133I, E/V136A, and Q139H, a polypeptide linker, and a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the TCRα constant domain.

In particular embodiments, a fusion polypeptide comprises a TCRα chain that comprises a constant domain comprising minimal murinization amino acid substitutions at positions 90, 91, 92, and 93, and hydrophobic amino acid substitutions at positions 115, 118, and 119 of the TCRα constant domain, wherein the amino acid sequence of the TCRα constant domain is at least 95%, 96%, 97%, 98%, or 99% identical to the amino acid sequence set forth in SEQ ID NO: 4, a polypeptide linker, and a TCRβ chain that comprises a constant domain comprising minimal murinization amino acid substitutions at positions 18, 22, 133, 136, and 139, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein comprises from 5′ to 3′, a TCRα chain that comprises a constant domain comprising minimal murinization amino acid substitutions at positions 90, 91, 92, and 93, and hydrophobic amino acid substitutions at positions 115, 118, and 119 of the TCRα constant domain, wherein the amino acid sequence of the TCRα constant domain is at least 95%, 96%, 97%, 98%, or 99% identical to the amino acid sequence set forth in SEQ ID NO: 4, a polypeptide linker, and a TCRβ chain that comprises a constant domain comprising minimal murinization amino acid substitutions at positions 18, 22, 133, 136, and 139, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein that comprises from 5′ to 3′, a TCRβ chain that comprises a constant domain comprising minimal murinization amino acid substitutions at positions 18, 22, 133, 136, and 139, wherein the amino acid sequence of the TCRβ constant domain is at least 95%, 96%, 97%, 98%, or 99% identical to the amino acid sequence set forth in SEQ ID NO: 5 or SEQ ID NO: 6, a polypeptide linker, and a TCRα chain that comprises a constant domain comprising minimal murinization amino acid substitutions at positions 90, 91, 92, and 93, and hydrophobic amino acid substitutions at positions 115, 118, and 119 of the TCRα constant domain, wherein the amino acid sequence of the TCRα constant domain is at least 95%, 96%, 97%, 98%, or 99% identical to the amino acid sequence set forth in SEQ ID NO: 4.

In particular embodiments, a fusion polypeptide comprises a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4, a polypeptide linker, and a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein comprises from 5′ to 3′, a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4, a polypeptide linker, and a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein that comprises from 5′ to 3′, a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6, a polypeptide linker, and a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4.

In preferred embodiments, the polypeptide linker is a polypeptide cleavage signal. Illustrative examples of polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 38), for example, ENLYFQG (SEQ ID NO: 39) and ENLYFQS (SEQ ID NO: 40), wherein X represents any amino acid (cleavage by TEV occurs between Q and G or Q and S).

In particular embodiments, the polypeptide cleavage signal is a viral self-cleaving peptide or ribosomal skipping sequence.

Illustrative examples of ribosomal skipping sequences include, but are not limited to: a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 1.

TABLE 1

| | |
|---|---|
| SEQ ID NO: 41 | GSGATNFSLLKQAGDVEENPGP |
| SEQ ID NO: 42 | ATNFSLLKQAGDVEENPGP |
| SEQ ID NO: 43 | LLKQAGDVEENPGP |
| SEQ ID NO: 44 | GSGEGRGSLLTCGDVEENPGP |
| SEQ ID NO: 45 | EGRGSLLTCGDVEENPGP |
| SEQ ID NO: 46 | LLTCGDVEENPGP |
| SEQ ID NO: 47 | GSGQCTNYALLKLAGDVESNPGP |
| SEQ ID NO: 48 | QCTNYALLKLAGDVESNPGP |
| SEQ ID NO: 49 | LLKLAGDVESNPGP |
| SEQ ID NO: 50 | GSGVKQTLNFDLLKLAGDVESNPGP |
| SEQ ID NO: 51 | VKQTLNFDLLKLAGDVESNPGP |
| SEQ ID NO: 52 | LLKLAGDVESNPGP |
| SEQ ID NO: 53 | LLNFDLLKLAGDVESNPGP |
| SEQ ID NO: 54 | TLNFDLLKLAGDVESNPGP |
| SEQ ID NO: 55 | LLKLAGDVESNPGP |
| SEQ ID NO: 56 | NFDLLKLAGDVESNPGP |
| SEQ ID NO: 57 | QLLNFDLLKLAGDVESNPGP |
| SEQ ID NO: 58 | APVKQTLNFDLLKLAGDVESNPGP |
| SEQ ID NO: 59 | VTELLYRMKRAETYCPRPLLAIHPTEARHKQKI |
| | VAPVKQT |
| SEQ ID NO: 60 | LNFDLLKLAGDVESNPGP |
| SEQ ID NO: 61 | LLAIHPTEARHKQKIVAPVKQTLNFDLLKLAGD |
| | VESNPGP |
| SEQ ID NO: 62 | EARHKQKIVAPVKQTLNFDLLKLAGDVESNPGP |

In particular embodiments, a fusion polypeptide comprises a minimally murinized TCRα chain, a polypeptide cleavage signal, and a minimally murinized TCRβ chain, wherein the TCRα chain transmembrane domain comprises hydrophobic amino acid substitutions. In some embodiments, a fusion protein that comprises from 5′ to 3′, a minimally murinized TCRα chain, a polypeptide cleavage signal, and a minimally murinized TCRβ chain, wherein the TCRα chain transmembrane domain comprises hydrophobic amino acid substitutions. In some embodiments, a fusion protein that comprises from 5′ to 3′, a minimally murinized TCRβ chain, a polypeptide cleavage signal, and a minimally murinized TCRα chain, wherein the TCRα chain transmembrane domain comprises hydrophobic amino acid substitutions.

In particular embodiments, a fusion polypeptide comprises a minimally murinized TCRα chain comprising 4 amino acid substitutions in the TCRα constant region, a polypeptide cleavage signal, and a minimally murinized TCRβ chain comprising 5 amino acid substitutions in the TCRβ constant region, wherein the TCRα chain transmembrane domain further comprises three hydrophobic amino acid substitutions. In some embodiments, a fusion protein comprises from 5′ to 3′, a minimally murinized TCRα chain comprising 4 amino acid substitutions in the TCRα constant region, a polypeptide cleavage signal, and a minimally murinized TCRβ chain comprising 5 amino acid substitutions in the TCRβ constant region, wherein the TCRα chain transmembrane domain further comprises three hydrophobic amino acid substitutions. In some embodiments, a fusion protein that comprises from 5′ to 3′, a minimally murinized TCRβ chain comprising 5 amino acid substitutions in the TCRβ constant region, a polypeptide cleavage signal, and a minimally murinized TCRα chain comprising 4 amino acid substitutions in the TCRα constant region, wherein the TCRα chain transmembrane domain further comprises three hydrophobic amino acid substitutions.

In particular embodiments, a fusion polypeptide comprises a minimally murinized TCRα chain comprising the following minimal murinization amino acid substitutions, P90S, E91D, S92V, and S93P and the following hydrophobic amino acid substitutions in the transmembrane domain, S115L, G118V, and F119L of the TCRα constant domain, a polypeptide cleavage signal, and a TCRβ chain that comprises a constant domain comprising the following minimal murinization amino acid substitutions, E18K, S22A, F133I, E/V136A, and Q139H. In some embodiments, a fusion protein comprises from 5′ to 3′, a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the TCRα constant domain, a polypeptide cleavage signal, and a TCRβ chain that comprises a constant domain comprising the following minimal murinization amino acid substitutions, E18K, S22A, F133I, E/V136A, and Q139H. In some embodiments, a fusion protein that comprises from 5′ to 3′, a TCRβ chain that comprises a constant domain comprising the following minimal murinization amino acid substitutions, E18K, S22A, F133I, E/V136A, and Q139H, a polypeptide cleavage signal, and a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the TCRα constant domain.

In particular embodiments, a fusion polypeptide comprises a TCRα chain that comprises a constant domain comprising minimal murinization amino acid substitutions at positions 90, 91, 92, and 93, and hydrophobic amino acid substitutions at positions 115, 118, and 119 of the TCRα constant domain, wherein the amino acid sequence of the TCRα constant domain is at least 95%, 96%, 97%, 98%, or 99% identical to the amino acid sequence set forth in SEQ ID NO: 4, a polypeptide cleavage signal, and a TCRβ chain that comprises a constant domain comprising minimal murinization amino acid substitutions at positions 18, 22, 133, 136, and 139, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein comprises from 5′ to 3′, a TCRα chain that comprises a constant domain comprising minimal murinization amino acid substitutions at positions 90, 91, 92, and 93, and hydrophobic amino acid substitutions at positions 115, 118, and 119 of the TCRα constant domain, wherein the amino acid sequence of the TCRα constant domain is at least 95%, 96%, 97%, 98%, or 99% identical to the amino acid sequence set forth in SEQ ID NO: 4, a polypeptide cleavage signal, and a TCRβ chain that comprises a constant domain comprising minimal murinization amino acid substitutions at positions 18, 22, 133, 136, and 139, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein that comprises from 5′ to 3′, a TCRβ chain that comprises a constant domain comprising minimal murinization amino acid substitutions at positions 18, 22, 133, 136, and 139, wherein the amino acid sequence of the TCRβ constant domain is at least 95%, 96%, 97%, 98%, or 99% identical to the amino acid sequence set forth in SEQ ID NO: 5 or SEQ ID NO: 6, a polypeptide cleavage signal, and a TCRα chain that comprises a constant domain comprising minimal murinization amino acid substitutions at positions 90, 91, 92, and 93, and hydrophobic amino acid substitutions at positions 115, 118, and 119 of the TCRα constant domain, wherein the amino acid sequence of the TCRα constant domain is at least 95%, 96%, 97%, 98%, or 99% identical to the amino acid sequence set forth in SEQ ID NO: 4.

In particular embodiments, a fusion polypeptide comprises a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4, a polypeptide cleavage signal, and a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein comprises from 5′ to 3′, a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4, a polypeptide cleavage signal, and a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6. In some embodiments, a fusion protein that comprises from 5′ to 3′, a TCRβ chain that comprises a constant domain comprising the following minimal murinization amino acid substitutions, E18K, S22A, F133I, E/V136A, and Q139H, wherein the amino acid sequence of the TCRβ constant domain is at least 95%, 96%, 97%, 98%, or 99% identical to the amino acid sequence set forth in SEQ ID NO: 5 or SEQ ID NO: 6, a polypeptide cleavage signal, and a TCRα chain that comprises a constant domain comprising the following minimal murinization amino acid substitutions, P90S, E91D, S92V, and S93P and the following hydrophobic amino acid substitutions in the transmembrane domain, S115L, G118V, and F119L of the constant domain, wherein the amino acid sequence of the TCRα constant domain is at least 95%, 96%, 97%, 98%, or 99% identical to the amino acid sequence set forth in SEQ ID NO: 4.

In particular embodiments, the fusion protein comprises a polypeptide cleavage signal that is a viral self-cleaving peptide or ribosomal skipping sequence.

In particular embodiments, the fusion protein comprises a polypeptide cleavage signal that is a viral 2A peptide.

In particular embodiments, the fusion protein comprises a polypeptide cleavage signal that is an aphthovirus 2A peptide, a potyvirus 2A peptide, or a cardiovirus 2A peptide.

In particular embodiments, the fusion protein comprises a polypeptide cleavage signal that is a viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

### F. Polynucleotides

In particular embodiments, one or more polynucleotides encoding one or more TCR polypeptides, TCRα chain polypeptides, TCRβ chain polypeptides, TCR fusion polypeptides, and fragments thereof is provided. As used herein, the terms “polynucleotide” or “nucleic acid” refer to deoxyribonucleic acid (DNA), ribonucleic acid (RNA) and DNA/RNA hybrids. Polynucleotides may be monocistronic or polycistronic, single-stranded or double-stranded, and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths, ” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%,76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99% or 100% sequence identity to a reference sequence.

Illustrative examples of polynucleotides include, but are not limited to polynucleotides encoding SEQ ID NOs: 4-26.

As used herein, “isolated polynucleotide” refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a recombinant polynucleotide, a semi-synthetic polynucleotide, or a polynucleotide obtained or derived from a recombinant source.

In various embodiments, a polynucleotide comprises an mRNA encoding a polypeptide contemplated herein. In certain embodiments, the mRNA comprises a cap, one or more nucleotides, and a poly(A) tail.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

Polynucleotide variants include polynucleotide fragments that encode biologically active polypeptide fragments or variants. As used herein, the term “polynucleotide fragment” refers to a polynucleotide fragment at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more nucleotides in length that encodes a polypeptide variant that retains at least 100%, at least 90%, at least 80%, at least 70%, at least 60%, at least 50%, at least 40%, at least 30%, at least 20%, at least 10%, or at least 5% of the naturally occurring polypeptide activity. Polynucleotide fragments refer to a polynucleotide that encodes a polypeptide that has an amino-terminal deletion, a carboxyl-terminal deletion, and/or an internal deletion or substitution of one or more amino acids of a naturally-occurring or recombinantly-produced polypeptide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette encodes one or more chains of a TCR. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide, polypeptide variant, or fusion polypeptide. A vector may comprise 1, 2, 3, 4, 5, 6, 7, 8, 9, or 10 polynucleotides-of-interest. In certain embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

Illustrative examples of vectors include, but are not limited to plasmid, autonomously replicating sequences, and transposable elements, e.g., piggyBac, Sleeping Beauty, Mosl, Tc1/mariner, To12, mini-To12, Tc3, MuA, Himar I, Frog Prince, and derivatives thereof.

Additional Illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to, pClneo vectors (Promega) for expression in mammalian cells; pLenti4N5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, vectors include, but are not limited to expression vectors and viral vectors, and will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) U3 promoter (Haas et al. Journal of Virology. 2003;77(17): 9439-9450).

In one embodiment, a vector comprises an MNDU3 promoter.

In one embodiment, a vector comprises an EF1a promoter comprising the first intron of the human EF1a gene.

In one embodiment, a vector comprises an EF1a promoter that lacks the first intron of the human EF1a gene.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO:63), where R is a purine (A or G) (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a desired polypeptide, e.g., a TCR.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art.

In some embodiments, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In particular embodiments, one or more polynucleotides encoding a TCRα chain and a TCRβ chain are introduced into a cell (e.g., an immune effector cell) by non-viral or viral vectors. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a T cell.

Illustrative examples of non-viral vectors include, but are not limited to mRNA, plasmids (e.g., DNA plasmids or RNA plasmids), transposons, cosmids, and bacterial artificial chromosomes.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

In various embodiments, the polynucleotide is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, viral vectors are used to deliver one or more polynucleotides contemplated herein to a T cell.

Illustrative examples of viral vector systems suitable for use in particular embodiments contemplated herein include but are not limited to adeno-associated virus (AAV), retrovirus (including lentivirus), herpes simplex virus, adenovirus, and vaccinia virus vectors.

In particular embodiments, a polycistronic polynucleotide encoding a TCR comprising a TCRα chain and a TCRβ chain is introduced into a cell by a non-viral or viral vector. In particular embodiments, a polycistronic polynucleotide encoding a fusion protein encoding a TCR contemplated herein comprising a minimally murinized TCRα chain and hydrophobic amino acid substitutions in the TCRα transmembrane domain, a polypeptide cleavage signal, and a minimally murinized TCRβ chain.

In particular embodiments, a polycistronic polynucleotide encoding a TCR comprising a TCRα chain and a TCRβ chain is introduced into a cell by a non-viral or viral vector. In particular embodiments, a polycistronic polynucleotide encoding a fusion protein encoding a TCR contemplated herein comprising a minimally murinized TCRα chain and hydrophobic amino acid substitutions in the TCRα transmembrane domain, an IRES, and a minimally murinized TCRβ chain.

In particular embodiments, the polycistronic polynucleotide comprises the TCRα chain 5′ to the TCRβ chain. In other embodiments, the polycistronic polynucleotide comprises the TCRα chain 3′ to the TCRβ chain.

### G. Genetically Modified Cells

In various embodiments, cells genetically modified to express a TCR contemplated herein comprising a minimally murinized TCRα chain and hydrophobic amino acid substitutions in the TCRα transmembrane domain, and a minimally murinized TCRβ chain for use in the treatment of cancer are provided. In various embodiments, immune effector cells genetically modified to express a TCR contemplated herein comprising a minimally murinized TCRα chain and hydrophobic amino acid substitutions in the TCRα transmembrane domain and a minimally murinized TCRβ chain are used in preparation or manufacture of a medicament for the treatment of cancer.

In particular embodiments, a polynucleotide encoding a TCR contemplated herein is introduced into immune effector cells so as express a TCR contemplated herein and to redirect the immune effector cells to target cells expressing a target antigen. In particular embodiments, one or more polynucleotides encoding a TCR contemplated herein comprising a minimally murinized TCRα chain and hydrophobic amino acid substitutions in the TCRα transmembrane domain and a minimally murinized TCRβ chain is introduced into one or more immune effector cells. In particular embodiments, a polynucleotide encoding a fusion protein comprising a TCR comprising a minimally murinized TCRα chain and hydrophobic amino acid substitutions in the TCRα transmembrane domain, a polypeptide linker, e.g., a polypeptide cleavage signal, and a minimally murinized TCRβ chain is introduced into one or more immune effector cells.

An “immune effector cell,” is any cell of the immune system that has one or more effector functions (e.g., cytotoxic cell killing activity, secretion of cytokines, induction of ADCC and/or CDC). Illustrative immune effector cells contemplated herein are T lymphocytes, including but not limited to cytotoxic T cells (CTLs; CD8⁺ T cells), TILs, and helper T cells (HTLs; CD4⁺ T cells. In a particular embodiment, the cells comprise αβ T cells. In a particular embodiment, the cells comprise γδ T cells modified to express an αβ TCR. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are autologous.

Illustrative immune effector cells used with the TCRs contemplated in particular embodiments include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Thl) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4⁻CD8⁻T cell, or any other subset of T cells. Other illustrative populations of T cells suitable for use in particular embodiments include naive T cells (T_{N}), T memory stem cells (T_{SCM}), central memory T cells (T_{CM}), effector memory T cells (T_{EM}), and effector T cells (T_{EFF}).

As would be understood by the skilled person, other cells may also be used as immune effector cells with the TCRs contemplated herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoietic stem cells (HSCs) contained within the CD34⁺ population of cells derived from cord blood, bone marrow or mobilized peripheral blood which upon administration in a subject differentiate into mature immune effector cells, or which can be induced in vitro to differentiate into mature immune effector cells.

The term, “CD34⁺ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34⁺ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Methods for making the immune effector cells that express a TCR contemplated herein are provided in particular embodiments. In one embodiment, the method comprises transfecting or transducing immune effector cells isolated from an individual such that the immune effector cells express a polycistronic message encoding a TCR comprising a modified TCRα chain and a modified TCRβ chain or a fusion protein encoding a TCR contemplated herein comprising a minimally murinized TCRα chain and hydrophobic amino acid substitutions in the TCRα transmembrane domain, a polypeptide linker, and a minimally murinized TCRβ chain.

In a preferred embodiment, the method comprises transfecting or transducing immune effector cells isolated from an individual such that the immune effector cells express a polycistronic message encoding a TCR contemplated herein comprising a minimally murinized TCRα chain and hydrophobic amino acid substitutions in the TCRα transmembrane domain, a 2A self-cleaving polypeptide, and a minimally murinized TCRβ chain. In particular embodiments, the transduced cells are subsequently cultured for expansion, prior to administration to a subject.

In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR contemplated herein comprising a minimally murinized TCRα chain and hydrophobic amino acid substitutions in the TCRα transmembrane domain, a linker, and a minimally murinized TCRβ chain. In this regard, the immune effector cells may be cultured before and/or after being genetically modified.

In particular embodiments, prior to in vitro manipulation or genetic modification of the immune effector cells described herein, the source of cells is obtained from a subject. In particular embodiments, modified immune effector cells comprise T cells.

In particular embodiments, PBMCs may be directly genetically modified to express a polycistronic message encoding a TCR contemplated herein comprising a minimally murinized TCRα chain and hydrophobic amino acid substitutions in the TCRα transmembrane domain, a polypeptide linker, and a minimally murinized TCRβ chain using methods contemplated herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are activated and stimulated for expansion and then genetically modified with the TCRs contemplated herein (e.g., transduced with a viral vector comprising a nucleic acid encoding a polycistronic message encoding a TCR contemplated herein comprising a minimally murinized TCRα chain and hydrophobic amino acid substitutions in the TCRα transmembrane domain, a polypeptide linker, and a minimally murinized TCRβ chain) and then are activated and expanded in vitro. In various embodiments, T cells can be activated and expanded before or after genetic modification, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In one embodiment, CD34⁺ cells are transduced with a nucleic acid construct contemplated herein. In certain embodiments, the transduced CD34⁺ cells differentiate into mature immune effector cells in vivo following administration into a subject, generally the subject from whom the cells were originally isolated. In another embodiment, CD34⁺ cells may be stimulated in vitro prior to exposure to or after being genetically modified with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In particular embodiments, a population of modified immune effector cells for the treatment of cancer comprises a CAR and CCR contemplated herein. For example, a population of modified immune effector cells are prepared from peripheral blood mononuclear cells (PBMCs) obtained from a patient diagnosed with B cell malignancy described herein (autologous donors). The PBMCs form a heterogeneous population of T lymphocytes that can be CD4⁺, CD8⁺, or CD4⁺ and CD8⁺.

### H. Compositions and Formulations

The compositions contemplated herein may comprise one or more TCR polypeptides, TCRα chain polypeptides, TCRβ chain polypeptides, TCR fusion polypeptides, polynucleotides, vectors comprising same, genetically modified immune effector cells, etc., as contemplated herein. Compositions include, but are not limited to pharmaceutical compositions. In preferred embodiments, a composition comprises one or more cells modified to express an engineered TCR comprising a minimally murinized TCRα chain and hydrophobic amino acid substitutions in the TCRα transmembrane domain, and a minimally murinized TCRβ chain or a fusion protein comprising a TCR comprising a minimally murinized TCRα chain and hydrophobic amino acid substitutions in the TCRα transmembrane domain, a polypeptide linker, e.g., a polypeptide cleavage signal, and a minimally murinized TCRβ chain. In preferred embodiments, a composition comprises one or more cells modified to express a fusion protein comprising a TCR comprising a minimally murinized TCRα chain and hydrophobic amino acid substitutions in the TCRα transmembrane domain, a 2A self-cleaving polypeptide, and a minimally murinized TCRβ chain.

A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy. In preferred embodiments, a pharmaceutical composition comprises a pharmaceutically acceptable carrier, diluent or excipient and one or more cells that have been modified to express an engineered TCR comprising a minimally murinized TCRα chain and hydrophobic amino acid substitutions in the TCRα transmembrane domain, and a minimally murinized TCRβ chain or a fusion protein comprising a TCR comprising a minimally murinized TCRα chain and hydrophobic amino acid substitutions in the TCRα transmembrane domain, a polypeptide linker, and a minimally murinized TCRβ chain.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes but is not limited toisotonic saline; Ringer's solution; ethyl alcohol; phosphate buffer solutions; and any other compatible substances employed in pharmaceutical formulations.

In particular embodiments, compositions comprise an amount of immune effector cells expressing a TCR comprising a minimally murinized TCRα chain and hydrophobic amino acid substitutions in the TCRα transmembrane domain, and a minimally murinized TCRβ chain. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cells effective to achieve the desired prophylactic result. Typically, but not necessarily, since a prophylactic dose is used in subjects prior to or at an earlier stage of disease, the prophylactically effective amount is less than the therapeutically effective amount.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions to be administered can be determined by a physician with consideration of individual differences in age, weight, tumor size, extent of infection or metastasis, and condition of the patient (subject).

It can generally be stated that a pharmaceutical composition comprising the T cells described herein may be administered at a dosage of 10⁶ to 10¹³ cells/kg body weight, preferably 10⁸ to 10¹³ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁶, 10⁷, 10⁸, 10⁹, 10¹⁰, 10¹¹, 10¹² or 10¹³ cells. Compositions may be administered multiple times at dosages within these ranges. The cells may be allogeneic, syngeneic, xenogeneic, or autologous to the patient undergoing therapy.

Compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, or isotonic sodium chloride. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising immune effector cells contemplated herein are formulated in a solution comprising PlasmaLyte A.

In another preferred embodiment, compositions comprising immune effector cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising immune effector cells contemplated herein are formulated in a solution comprising 50:50 PlasmaLyte A to CryoStor CS10.

In a particular embodiment, compositions comprise an effective amount of genome edited immune effector cells modified to express a TCR comprising a minimally murinized TCRα chain and hydrophobic amino acid substitutions in the TCRα transmembrane domain, and a minimally murinized TCRβ chain, alone or in combination with one or more therapeutic agents. Thus, the immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated in particular embodiments include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising genome edited immune effector cells modified to express a TCR comprising a minimally murinized TCRα chain and hydrophobic amino acid substitutions in the TCRα transmembrane domain, and a minimally murinized TCRβ chain may be administered in conjunction with any number of chemotherapeutic agents.

In particular embodiments, a composition comprising immune effector modified to express a TCR comprising a minimally murinized TCRα chain and hydrophobic amino acid substitutions in the TCRα transmembrane domain, and a minimally murinized TCRβ chain is administered with a therapeutic antibody. Illustrative examples of therapeutic antibodies suitable for combination with the CAR modified T cells contemplated in particular embodiments, include but are not limited to, atezolizumab, avelumab, bavituximab, bevacizumab (avastin), bivatuzumab, blinatumomab, conatumumab, crizotinib, daratumumab, duligotumab, dacetuzumab, dalotuzumab, durvalumab, elotuzumab (HuLuc63), gemtuzumab, ibritumomab, indatuximab, inotuzumab, ipilimumab, lorvotuzumab, lucatumumab, milatuzumab, moxetumomab, nivolumab, ocaratuzumab, ofatumumab, pembrolizumab, rituximab, siltuximab, teprotumumab, and ublituximab.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22^{nd} Edition. Edited by Loyd V. Allen Jr. Philadelphia, Pa.: Pharmaceutical Press; 2012, which is incorporated by reference herein, in its entirety.

### I. Therapeutic Methods

The genetically modified immune effector cells expressing a TCR comprising a minimally murinized TCRα chain and hydrophobic amino acid substitutions in the TCRα transmembrane domain, and a minimally murinized TCRβ chain contemplated herein provide improved methods of adoptive immunotherapy for use in the prevention, treatment, and amelioration cancers or for preventing, treating, or ameliorating at least one symptom associated with cancer.

In one embodiment, a type of cellular therapy where T cells are genetically modified to express a TCR comprising a minimally murinized TCRα chain and hydrophobic amino acid substitutions in the TCRα transmembrane domain, and a minimally murinized TCRβ chain are infused to a recipient in need thereof is provided. The infused cell is able to kill disease causing cells in the recipient. Unlike antibody therapies, T cell therapies are able to replicate in vivo resulting in long-term persistence that can lead to sustained cancer therapy.

In one embodiment, T cells that express a TCR comprising a minimally murinized TCRα chain and hydrophobic amino acid substitutions in the TCRα transmembrane domain, and a minimally murinized TCRβ chain can undergo robust in vivo T cell expansion and can persist for an extended amount of time. In another embodiment, T cells that express a TCR comprising a minimally murinized TCRα chain and hydrophobic amino acid substitutions in the TCRα transmembrane domain, and a minimally murinized TCRβ 5 chain evolve into specific memory T cells or stem cell memory T cells that can be reactivated to inhibit any additional tumor formation or growth.

In particular embodiments, modified immune effector cells that express a TCR comprising a minimally murinized TCRα chain and hydrophobic amino acid substitutions in the TCRα transmembrane domain, and a minimally murinized TCRβ chain contemplated herein are used in the treatment of solid tumors or cancers.

In particular embodiments, the modified immune effector cells contemplated herein are used in the treatment of solid tumors or cancers including, but not limited to: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In particular embodiments, the modified immune effector cells contemplated herein are used in the treatment of solid tumors or cancers including, without limitation, non-small cell lung carcinoma, head and neck squamous cell carcinoma, colorectal cancer, pancreatic cancer, breast cancer, thyroid cancer, bladder cancer, cervical cancer, esophageal cancer, ovarian cancer, gastric cancer endometrial cancer, gliomas, glioblastomas, and oligodendroglioma.

In particular embodiments, the modified immune effector cells contemplated herein are used in the treatment of solid tumors or cancers including, without limitation, non-small-cell lung cancer, metastatic colorectal cancer, glioblastoma, head and neck cancer, pancreatic cancer, and breast cancer.

In particular embodiments, the modified immune effector cells contemplated herein are used in the treatment of glioblastoma.

In particular embodiments, the modified immune effector cells that express a TCR comprising a minimally murinized TCRα chain and hydrophobic amino acid substitutions in the TCRα transmembrane domain, and a minimally murinized TCRβ chain contemplated herein are used in the treatment of liquid cancers or hematological cancers.

In particular embodiments, the modified immune effector cells contemplated herein are used in the treatment of B-cell malignancies, including but not limited to: leukemias, lymphomas, and multiple myeloma.

In particular embodiments, the modified immune effector cells contemplated herein are used in the treatment of liquid cancers including, but not limited to leukemias, lymphomas, and multiple myelomas: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CIVIL), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In particular embodiments, the modified immune effector cells contemplated herein are used in the treatment of acute myeloid leukemia (AML).

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In preferred embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein, e.g., genetically modified T cells that express a TCR comprising a minimally murinized TCRα chain and hydrophobic amino acid substitutions in the TCRα transmembrane domain, and a minimally murinized TCRβ chain, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a similar physiological response (i.e., downstream effects) in a cell, as compared to the response caused by either vehicle, a control molecule/composition, or the response in a particular cell lineage. A comparable response is one that is not significantly different or measurable different from the reference response.

In one embodiment, a method of treating cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of immune effector cells, e.g., T cells that express a TCR comprising a minimally murinized TCRα chain and hydrophobic amino acid substitutions in the TCRα transmembrane domain, and a minimally murinized TCRβ chain, in the composition administered to a subject is at least 1×10⁷ cells, at least 0.5×10⁸ cells, at least 1×10⁸ cells, at least 0.5×10⁹ cells, at least 1×10⁹ cells, at least 1×10¹⁰ cells, at least 1×10¹¹ cells, at least 1×10¹² cells, at least 5×10¹² cells, or at least 1×10¹³ cells.

In particular embodiments, about 1×10⁷ T cells to about 1×10¹³ T cells, about 1×10⁸ T cells to about 1×10¹³ T cells, about 1×10⁹ T cells to about 1×10¹³ T cells, about 1×10¹⁰ T cells to about 1×10¹³ T cells, about 1×10¹¹ T cells to about 1×10¹³ T cells, or about 1×10¹² T cells to about 1×10¹³ T cells are administered to a subject.

In one embodiment, the amount of immune effector cells, e.g., T cells that express a TCR comprising a minimally murinized TCRα chain and hydrophobic amino acid substitutions in the TCRα transmembrane domain, and a minimally murinized TCRβ chain, in the composition administered to a subject is at least 0.1×10⁴ cells/kg of bodyweight, at least 0.5×10⁴ cells/kg of bodyweight, at least 1×10⁴cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In particular embodiments, about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight are administered to a subject.

One of ordinary skill in the art would recognize that multiple administrations of the compositions contemplated herein may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated immune effector cells to a subject and then subsequently redraw blood (or have an apheresis performed), activate immune effector cells therefrom, and reinfuse the patient with these activated and expanded immune effector cells. This process can be carried out multiple times every few weeks. In certain embodiments, immune effector cells can be activated from blood draws of from 10 cc to 400 cc. In certain embodiments, immune effector cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a B cell related condition in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In one embodiment, a method of treating a subject diagnosed with a cancer is provided comprising removing immune effector cells from the subject, genetically modifying said immune effector cells with a vector comprising a nucleic acid encoding a TCR comprising a minimally murinized TCRα chain and hydrophobic amino acid substitutions in the TCRα transmembrane domain, a polypeptide linker, and a minimally murinized TCRβ chain contemplated herein, thereby producing a population of modified immune effector cells, and administering the population of modified immune effector cells to the same subject. In a preferred embodiment, the immune effector cells comprise T cells.

In certain embodiments, methods for stimulating an immune effector cell mediated immune modulator response to a target cell population in a subject are provided comprising the steps of administering to the subject an immune effector cell population expressing a nucleic acid construct encoding a TCR comprising a minimally murinized TCRα chain and hydrophobic amino acid substitutions in the TCRα transmembrane domain, a polypeptide linker, and a minimally murinized TCRβ chain contemplated herein.

The methods for administering the cell compositions contemplated in particular embodiments includes any method which is effective to result in reintroduction of ex vivo genetically modified immune effector cells that either directly express a TCR comprising a minimally murinized TCRα chain and hydrophobic amino acid substitutions in the TCRα transmembrane domain, a polypeptide linker, and a minimally murinized TCRβ chain contemplated herein in the subject or on reintroduction of the genetically modified progenitors of immune effector cells that on introduction into a subject differentiate into mature immune effector cells that express the TCR. One method comprises transducing peripheral blood T cells ex vivo with a nucleic acid construct contemplated herein and returning the transduced cells into the subject.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

Amino Acid Substitutions in T Cell Receptor (TCR) Constant Regions Synergistically Increase TCR Expression

MAGEA4 TCR sequences were cloned into lentiviral vectors using standard cloning techniques and modified. The TCR^{WT} construct is the non-modified parent construct. The TCR^{TM} construct contains three hydrophobic amino acid substitutions (S115L, G118V, F119L; numbered with reference to TCRα constant region) in the TCRα chain transmembrane domain. The TCR construct contains four murinizing amino acid substitutions (P90S, E91D, S92V, S93P; numbered with reference to TCRα constant region) in the TCRα chain constant region and also contains five murinizing amino acid substitutions (E18K, S22A, F133I, E/V136A, Q139H; numbered with reference to TCRβ constant region) in the TCRβ chain constant region. The TCRTM/^{MM} construct contains the nine murinizing amino acid substitutions in the TCRα and TCRβ chain constant regions as well as the three hydrophobic amino acid substitutions in the TCRα chain transmembrane domain.

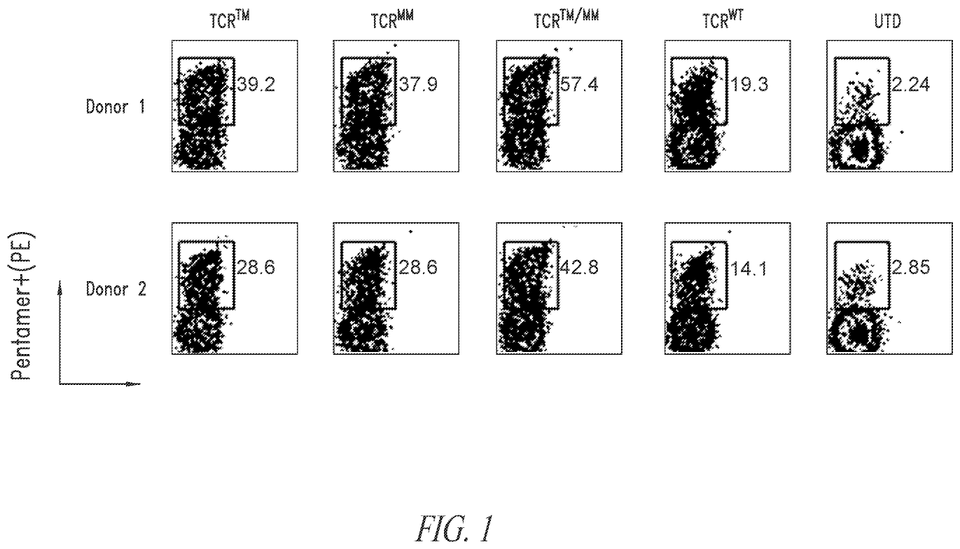

Peripheral blood mononuclear cells (PBMCs) from two normal donors were activated using CD3 and CD28 antibodies and transduced with lentiviral vectors encoding TCR^{WT}, TCR^{TM}, TCR^{MM}, or TCR^{TM/MM} and cultured for 10 days. After 10 days, UTD T cells or T cells transduced with lentiviral vectors encoding TCR^{WT}, TCR^{TM}, TCR^{MM}, or TCR^{TM/MM} were stained with MAGEA4 pentamer-peptide labelling reagent at 1:20 dilution in flow staining buffer, and analyzed on flow cytometer for PE fluoresence. Flow analysis showed a 2-fold increase in TCR expression in T cells transduced with TCR^{TM} and TCR^{MM} lentiviral vectors and a synergistic 4-fold increase in T cells transduced with the lentiviral vector encoding TCR^{TM/MM} compared to T cells transduced with the lentiviral vector encoding TCR^{WT}. FIG. 1. T cells transduced with lentiviral vectors showed equivalent vector copy numbers (VCNs).

### Example 2

### T Cells Transduced with Modified TCRS Have Increased Expression Compared to T Cells Transduced with Unmodified TCR

PBMCs from two normal donors were activated using CD3 and CD28 antibodies and transduced using two different transduction conditions (Tdxn 1—enhanced tdxn process; Tdxn 2-base tdxn process) with lentiviral vectors encoding TCR^{WT} or TCR^{TM/MM} and cultured for 10 days. Untransduced (UTD) T cells were used as a control.

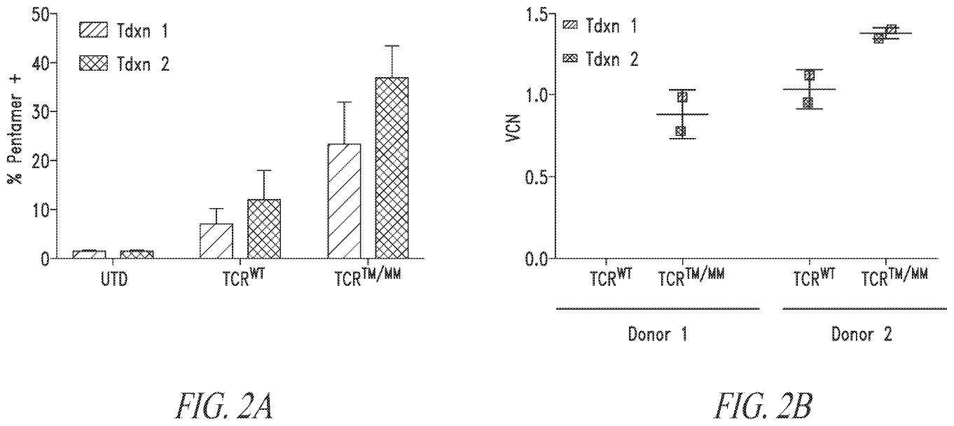

After 10 days, the cells were stained with MAGEA4 pentamer-peptide labelling reagent at 1:20 dilution in flow staining buffer, and analyzed on flow cytometer for PE fluorescence. Flow analysis showed a 4-fold increase in T cells transduced with the lentiviral vector encoding TCR^{TM/MM} compared to T cells transduced with the lentiviral vector encoding TCR^{WT} in both transduction conditions. FIG. 2A.

RT-PCR was used to measure vector copy number (VCN) and assess LVV integration under each transduction condition. VCNs were comparable under each transduction condition. FIG. 2B.

### Example 3

### T Cells Transduced with Modified TCRS Eliminate TCR Mis-Pairing

PBMCs were activated using CD3 and CD28 antibodies and transduced with lentiviral vectors encoding TCR^{WT} or TCR^{TM/MM} and cultured for 10 days.

After 10 days, TCR^{WT} and TCR^{TM/MM} transduced T cells were assessed for specific TCR pairing using dual staining with pentamer-peptide labeling PE reagent at 1:20 dilution and v-beta chain FITC labeling flourophore at 1:100 dilution in flow staining buffer. Specifically paired TCRs are indicated where the percentage positive cells detected by v-beta staining and tetramer antigen staining were equal.

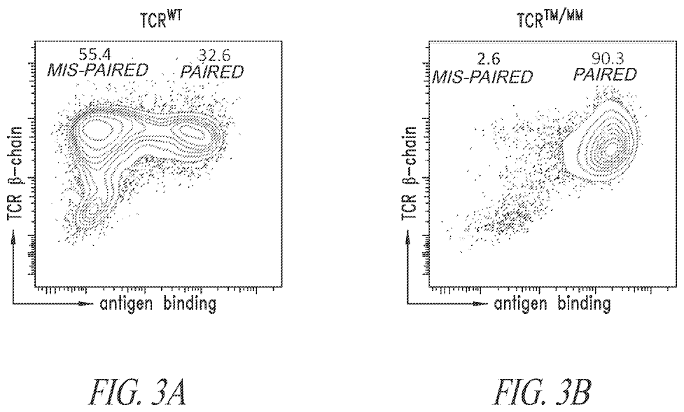

TCR^{TM/MM} transduced T cells showed >90% specific pairing compared to T cells transduced with TCR^{WT}. These data indicate that TCR mis-pairing was eliminated by the modifications present in TCR^{TM/MM}. FIG. 3A and FIG. 3B.

### Example 4

T Cells Transduced with Modified TCRS Possess Potent Anti-Tumor Properties

PBMCs from two normal donors were activated using CD3 and CD28 antibodies and transduced with lentiviral vectors encoding TCR^{WT} or TCR^{TM/MM} and cultured for 10 days.

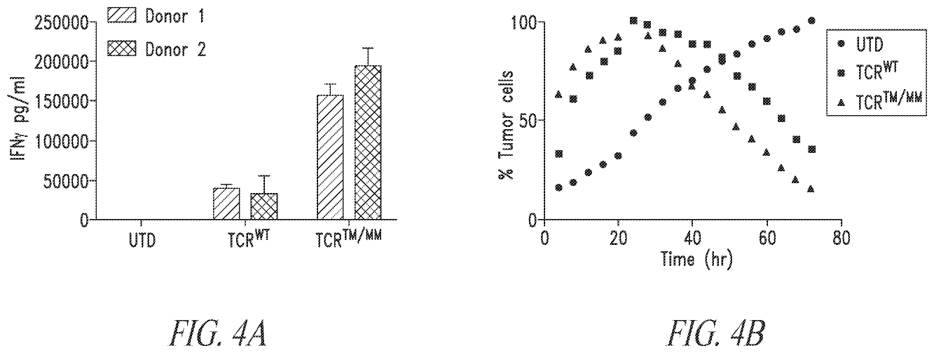

IFNγ Assays: UTD T cells or T cells transduced with lentiviral vectors encoding TCR^{WT} or TCR^{TM/MM} were co-cultured for 24 hours with MAGEA4 positive A549 tumor Nuc-red cells at an E:T ratio of 1:1 normalized based on TCR expression. After 24 h, supernantant was collected from these samples and analyzed using Meso Scale Discovery (MSD) assay to measure cytokine production. TCR^{TM/MM}-expressing T cells showed a significant (4-fold) increase in IFNy production compared to TCR^{WT}-expressing T cells. FIG. 4A.

Cytotoxicity Assays: UTD T cells or T cells transduced with lentiviral vectors encoding TCR^{WT} or TCR^{TM/MM} were co-cultured with MAGEA4 positive A549 tumor Nuc-red cells at an E:T ratio of 1:1 normalized based on TCR expression. Cytotoxicity was monitored over three days using an Incucyte S3. TCR^{TM/MM}-expressing T cells showed a steeper killing curve compared to TCR^{WT}-expressing T cells or UTD T cells. FIG. 4B.

### Example 5

Amino Acid Substitutions in T Cell Receptor (TCR) Constant Regions Synergistically Increase TCR Expression and Specific TCR Pairing in NY-ESO-1 TCRS

NY-ESO-1 TCR sequences (SEQ ID NOs: 15 and 16) were cloned into lentiviral vectors using standard cloning techniques and modified. The TCR^{MM} construct contains four murinizing amino acid substitutions (P90S, E91D, S92V, S93P; numbered with reference to TCRα constant region) in the TCRα chain constant region and also contains five murinizing amino acid substitutions (E18K, S22A, F133I, EN136A, Q139H; numbered with reference to TCRβ constant region) in the TCRβ chain constant region. The TCRTM/^{MM} construct contains the nine murinizing amino acid substitutions in the TCRα and TCRβ chain constant regions as well as three hydrophobic amino acid substitutions (S115L, G118V, F119L; numbered with reference to TCRα constant region) in the TCRα chain transmembrane domain.

Peripheral blood mononuclear cells (PBMCs) from two normal donors were activated using CD3 and CD28 antibodies and transduced with lentiviral vectors encoding TCR^{WT}, TCR^{MM}, or TCR^{TM/MM} and cultured for 10 days.

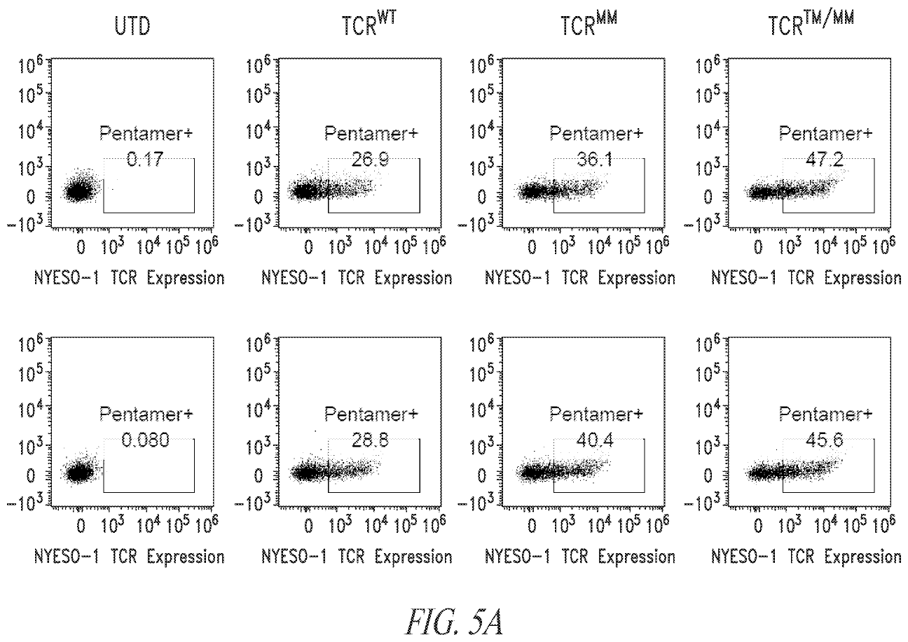

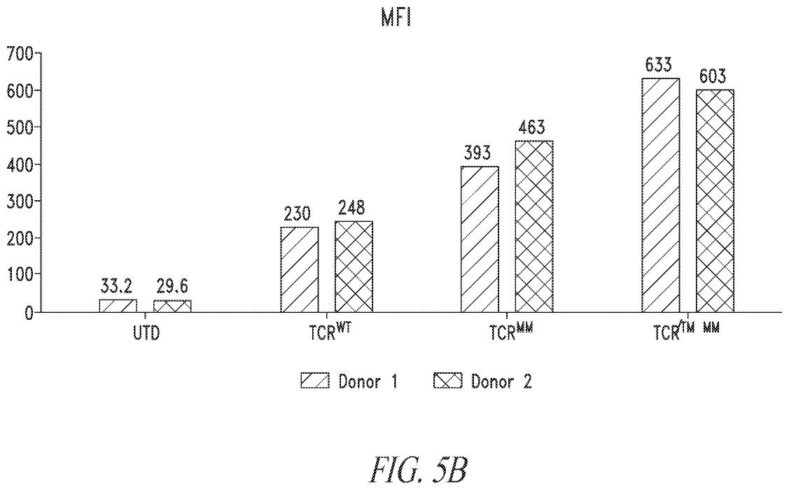

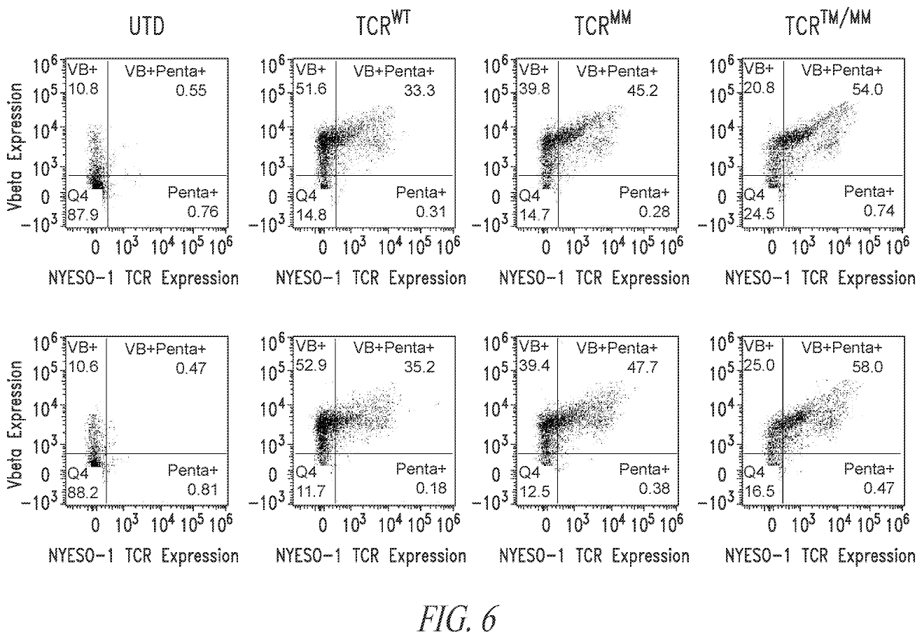

Expression: After 10 days, UTD T cells or cells transduced with the lentiviral vectors encoding TCR^{WT}, TCR^{MM}, or TCR^{TM/MM} were stained with NY-ESO-1 pentamer-peptide labelling reagent at 1:20 dilution in flow staining buffer, and analyzed on flow cytometer for PE fluoresence. Flow analysis showed increased TCR expression in T cells transduced with the lentiviral vector encoding TCR^{TM/MM} compared to UTD T cells or T cells transduced with the lentiviral vector encoding TCR^{WT} or TCR. FIG. 5A. Mean Flourescence Intensity of TCR staining in each donor is shown in FIG. 5B.

Pairing: After 10 days, UTD T cells or cells transduced with the lentiviral vectors encoding TCR^{WT}, TCR^{MM}, or TCR^{TM/MM} were assessed for specific TCR pairing using dual staining with NY-ESO-1 pentamer-peptide labeling PE reagent at 1:20 dilution and v-beta chain FITC labeling flourophore at 1:100 dilution in flow staining buffer. Specifically paired TCRs are indicated where the percentage positive cells detected by v-beta staining and tetramer antigen staining were equal. TCR^{TM/MM} transduced T cells showed increased specific pairing compared to UTD T cells or T cells transduced with the lentiviral vector encoding TCR^{WT} or TCR^{MM}. FIG. 6.

### Example 6

### Amino Acid Substitutions in T Cell Receptor (TCR) Constant Regions in MART-1 TCRs

MART-1 TCR a chain and f3 chain sequences (SEQ ID NOs: 7 and 8; SEQ ID NOs: 9 and 10) with enhanced pairing mutations (TCR^{TM/MM}) were generated and will be cloned into lentiviral vectors using standard cloning techniques. The TCR^{TM/MM} contains four murinizing amino acid substitutions (P90S, E91D, S92V, S93P; numbered with reference to TCRα constant region) and three hydrophobic amino acid substitutions (S115L, G118V, F119L; numbered with reference to TCRα constant region) in the TCRα chain constant region and also contains five murinizing amino acid substitutions (E18K, S22A, F133I, E/V136A, Q139H; numbered with reference to TCRβ constant region) in the TCRβ chain constant region.

Peripheral blood mononuclear cells (PBMCs) from two normal donors will be activated using CD3 and CD28 antibodies and transduced with lentiviral vectors encoding TCR^{WT} or TCR^{TM/MM} and cultured for 10 days.

Expression: After 10 days, UTD T cells or cells transduced with the lentiviral vectors encoding TCR^{WT} or TCR^{TM/MM} will be stained with MART-1 pentamer-peptide labelling reagent at 1:20 dilution in flow staining buffer, and analyzed on flow cytometer for PE fluoresence.

Pairing: After 10 days, UTD T cells or cells transduced with the lentiviral vectors encoding TCR^{WT} or TCR^{TM/MM} will be assessed for specific TCR pairing using dual staining with MART-1 pentamer-peptide labeling PE reagent at 1:20 dilution and v-beta chain FITC labeling flourophore at 1:100 dilution in flow staining buffer.

### Example 7

### Amino Acid Substitutions in T Cell Receptor (TCR) Constant Regions in WT-1 TCRS

WT-1 TCR α chain and β chain sequences (SEQ ID NOs: 11 and 12) with enhanced pairing mutations (TCR^{TM/MM}) were generated and will be cloned into lentiviral vectors using standard cloning techniques. The TCR^{TM/MM} contains four murinizing amino acid substitutions (P90S, E91D, S92V, S93P; numbered with reference to TCRα constant region) and three hydrophobic amino acid substitutions (S115L, G118V, F119L; numbered with reference to TCRα constant region) in the TCRα chain constant region and also contains five murinizing amino acid substitutions (E18K, S22A, F133I, EN136A, Q139H; numbered with reference to TCRβ constant region) in the TCRβ chain constant region.

Peripheral blood mononuclear cells (PBMCs) from two normal donors will be activated using CD3 and CD28 antibodies and transduced with lentiviral vectors encoding TCR^{WT} or TCR^{TM/MM} and cultured for 10 days.

Expression: After 10 days, UTD T cells or cells transduced with the lentiviral vectors encoding TCR^{WT} or TCR^{TM/MM} will be stained with WT-1 pentamer-peptide labelling reagent at 1:20 dilution in flow staining buffer, and analyzed on flow cytometer for PE fluoresence.

Pairing: After 10 days, UTD T cells or cells transduced with the lentiviral vectors encoding TCR^{WT} or TCR^{TM/MM} will be assessed for specific TCR pairing using dual staining with WT-1 pentamer-peptide labeling PE reagent at 1:20 dilution and v-beta chain FITC labeling flourophore at 1:100 dilution in flow staining buffer.

### Example 8

### Amino Acid Substitutions in T Cell Receptor (TCR) Constant Regions in HPV16 E6 TCRS

HPV16 E6 TCR α chain and β chain sequences (SEQ ID NOs: 13 and 14) with enhanced pairing mutations (TCR^{TM/MM}) were generated and will be cloned into lentiviral vectors using standard cloning techniques. The TCR^{TM/MM} contains four murinizing amino acid substitutions (P90S, E91D, S92V, S93P; numbered with reference to TCRα constant region) and three hydrophobic amino acid substitutions (S115L, G118V, F119L; numbered with reference to TCRα constant region) in the TCRα chain constant region and also contains five murinizing amino acid substitutions (E18K, S22A, F133I, E/V136A, Q139H; numbered with reference to TCRβ constant region) in the TCRβ chain constant region.

Peripheral blood mononuclear cells (PBMCs) from two normal donors will be activated using CD3 and CD28 antibodies and transduced with lentiviral vectors encoding TCR^{WT} or TCR^{TM/MM} and cultured for 10 days.

Expression: After 10 days, UTD T cells or cells transduced with the lentiviral vectors encoding TCRW^{T} or TCR^{TM/MM} will be stained with HPV16 E6 pentamer-peptide labelling reagent at 1:20 dilution in flow staining buffer, and analyzed on flow cytometer for PE fluoresence.

Pairing: After 10 days, UTD T cells or cells transduced with the lentiviral vectors encoding TCRW^{T} or TCR^{TM/MM} will be assessed for specific TCR pairing using dual staining with HPV16 E6 pentamer-peptide labeling PE reagent at 1:20 dilution and v-beta chain FITC labeling flourophore at 1:100 dilution in flow staining buffer.

### Example 9

### Amino Acid Substitutions in T Cell Receptor (TCR) Constant Regions in NY-ESO-1 TCRs

NY-ESO-1 TCR α chain and β0 chain sequences (SEQ ID NOs: 17 and 18; SEQ ID NOs: 19 and 20; SEQ ID NOs: 21 and 22) with enhanced pairing mutations (TCR^{TM/MM}) were generated and will be cloned into lentiviral vectors using standard cloning techniques. The TCR^{TM/MM} contains four murinizing amino acid substitutions (P90S, E91D, S92V, S93P; numbered with reference to TCRα constant region) and three hydrophobic amino acid substitutions (S115L, G118V, F119L; numbered with reference to TCRα constant region) in the TCRα chain constant region and also contains five murinizing amino acid substitutions (E18K, S22A, F133I, E/V136A, Q139H; numbered with reference to TCRβ constant region) in the TCRβ chain constant region.

Peripheral blood mononuclear cells (PBMCs) from two normal donors will be activated using CD3 and CD28 antibodies and transduced with lentiviral vectors encoding TCR^{WT} or TCR^{TM/MM} and cultured for 10 days.

Expression: After 10 days, UTD T cells or cells transduced with the lentiviral vectors encoding TCR^{WT} or TCR^{TM/MM} will be stained with NY-ESO-1 pentamer-peptide labelling reagent at 1:20 dilution in flow staining buffer, and analyzed on flow cytometer for PE fluoresence.

Pairing: After 10 days, UTD T cells or cells transduced with the lentiviral vectors encoding TCR^{WT} or TCR^{TM/MM} will be assessed for specific TCR pairing using dual staining with NY-ESO-1 pentamer-peptide labeling PE reagent at 1:20 dilution and v-beta chain FITC labeling flourophore at 1:100 dilution in flow staining buffer.

### Example 10

### Amino Acid Substitutions in T Cell Receptor (TCR) Constant Regions in HPV16 E7 TCRs

HPV16 E7 TCR α chain and β chain sequences (SEQ ID NOs: 23 and 24) with enhanced pairing mutations (TCR^{TM/MM}) were generated and will be cloned into lentiviral vectors using standard cloning techniques. The TCR^{TM/MM} contains four murinizing amino acid substitutions (P90S, E91D, S92V, S93P; numbered with reference to TCRα constant region) and three hydrophobic amino acid substitutions (S115L, G118V, F119L; numbered with reference to TCRα constant region) in the TCRα chain constant region and also contains five murinizing amino acid substitutions (E18K, S22A, F133I, E/V136A, Q139H; numbered with reference to TCRβ constant region) in the TCRβ chain constant region.

Peripheral blood mononuclear cells (PBMCs) from two normal donors will be activated using CD3 and CD28 antibodies and transduced with lentiviral vectors encoding TCR^{WT} or TCR^{TM/MM} and cultured for 10 days.

Expression: After 10 days, UTD T cells or cells transduced with the lentiviral vectors encoding TCR^{WT} or TCR^{TM/MM} will be stained with HPV16 E7 pentamer-peptide labelling reagent at 1:20 dilution in flow staining buffer, and analyzed on flow cytometer for PE fluoresence.

Pairing: After 10 days, UTD T cells or cells transduced with the lentiviral vectors encoding TCR^{WT} or TCR^{TM/MM} will be assessed for specific TCR pairing using dual staining with HPV16 E7 pentamer-peptide labeling PE reagent at 1:20 dilution and v-beta chain FITC labeling flourophore at 1:100 dilution in flow staining buffer.

### Example 11

### Amino Acid Substitutions in T Cell Receptor (TCR) Constant Regions in GP100 TCRs

GP100 TCR α chain and β chain sequences (SEQ ID NOs: 25 and 26) with enhanced pairing mutations (TCR^{TM/MM}) were generated and will be cloned into lentiviral vectors using standard cloning techniques. The TCR^{TM/MM} contains four murinizing amino acid substitutions (P90S, E91D, S92V, S93P; numbered with reference to TCRα constant region) and three hydrophobic amino acid substitutions (S115L, G118V, F119L; numbered with reference to TCRα constant region) in the TCRα chain constant region and also contains five murinizing amino acid substitutions (E18K, S22A, F133I, EN136A, Q139H; numbered with reference to TCRβ constant region) in the TCRβ chain constant region.

Peripheral blood mononuclear cells (PBMCs) from two normal donors will be activated using CD3 and CD28 antibodies and transduced with lentiviral vectors encoding TCR^{WT} or TCR^{TM/MM} and cultured for 10 days.

Expression: After 10 days, UTD T cells or cells transduced with the lentiviral vectors encoding TCR^{WT} or TCR^{TM/MM} will be stained with GP100 pentamer-peptide labelling reagent at 1:20 dilution in flow staining buffer, and analyzed on flow cytometer for PE fluoresence.

Pairing: After 10 days, UTD T cells or cells transduced with the lentiviral vectors encoding TCR^{WT} or TCR^{TM/MM} will be assessed for specific TCR pairing using dual staining with GP100 pentamer-peptide labeling PE reagent at 1:20 dilution and v-beta chain FITC labeling flourophore at 1:100 dilution in flow staining buffer.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An isolated T cell receptor (TCR) comprising a minimally murinized TCRα chain and a minimally murinized TCRβ chain, and wherein the TCRα chain transmembrane domain comprises hydrophobic amino acid substitutions, wherein the TCR does not bind MAGEA4.

2. An isolated T cell receptor (TCR) comprising:
(a) a TCRα chain that comprises a constant domain comprising minimal murinization amino acid substitutions at positions 90, 91, 92, and 93, and hydrophobic amino acid substitutions at positions 115, 118, and 119; and
(b) a TCRβ chain that comprises a constant domain comprising minimal murinization amino acid substitutions at positions 18, 22, 133, 136, and 139.

3. An isolated T cell receptor (TCR) comprising:
(a) a TCRα chain that comprises a constant domain comprising the amino acid substitutions, P90S, E91D, S92V, S93P, S115L, G118V, and F119L; and
(b) a TCRβ chain that comprises a constant domain comprising the amino acid substitutions E18K, S22A, F133I, E/V136A, and Q139H.

4. An isolated T cell receptor (TCR) comprising:
(a) a TCRα chain that comprises a constant domain comprising at least 4 minimal murinization amino acid substitutions and at least 3 hydrophobic amino acid substitutions in the TCRα chain transmembrane domain, wherein the TCRα chain constant domain comprises an amino acid sequence that is at least 95%, 96%, 97%, 98%, or 99% identical to the amino acid sequence set forth in SEQ ID NO: 4; and
(b) a TCRβ chain that comprises a constant domain comprising at least 5 minimal murinization amino acid substitutions, wherein the TCRβ chain constant domain comprises an amino acid sequence that is at least 95%, 96%, 97%, 98%, or 99% identical to the amino acid sequence set forth in SEQ ID NO: 5 or SEQ ID NO: 6.

5. An isolated T cell receptor (TCR) comprising:
(a) a TCRα chain comprising a constant domain comprising the amino acid sequence set forth in SEQ ID NO: 4; and
(b) a TCRβ chain comprising a constant domain comprising the amino acid sequence set forth in SEQ ID NO: 5 or SEQ ID NO: 6.

6. The isolated TCR of any one of the preceding claims, wherein the TCR binds a target antigen selected from the group consisting of: a-fetoprotein (AFP), B Melanoma Antigen (BAGE) family members, Brother of the regulator of imprinted sites (BORIS), Cancer-testis antigens, Cancer-testis antigen 83 (CT-83), Carbonic anhydrase IX (CA1X), Carcinoembryonic antigen (CEA), Cytomegalovirus (CMV) antigens, Cytotoxic T cell (CTL)-recognized antigen on melanoma (CAMEL), Epstein-Barr virus (EBV) antigens, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human Epidermal Growth Factor Receptor 2 (HER-2), Human papillomavirus (HPV)-E6, HPV-E7, Human telomerase reverse transcriptase (hTERT), Latent membrane protein 2 (LMP2), Melanoma antigen family A, 1 (MAGE-A1), MAGE-A2, MAGE-A3, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, Placenta-specific 1 (PLAC1), Preferentially expressed antigen in melanoma (PRAME), Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, Tyrosinase, Tyrosinase related protein (TRP)1, TRP2, Wilms tumor protein (WT-1), X Antigen Family Member 1 (XAGE1), and X Antigen Family Member 2 (XAGE2).

7. The isolated TCR of any one of claims 1 to 6, wherein the TCR expression and avidity is increased compared to a TCR that comprises a minimally murinized TCRα chain and a minimally murinized TCRβ chain but wherein the TCRα chain transmembrane domain does not comprise hydrophobic amino acid substitutions.

8. The isolated TCR of any one of claims 1 to 6, wherein the TCR expression and avidity is increased compared to a TCR that does not comprise a minimally murinized TCRα chain and a minimally murinized TCRβ chain but wherein the TCRα chain transmembrane domain comprises hydrophobic amino acid substitutions.

9. A fusion protein comprising the TCR α chain and the TCR β chain set forth in any one of the preceding claims.

10. A fusion protein comprising a minimally murinized TCRα chain wherein the TCRα chain transmembrane domain comprises hydrophobic amino acid substitutions; a polypeptide cleavage signal; and a minimally murinized TCRβ chain, wherein the fusion protein does not bind MAGEA4.

11. A fusion protein comprising:
(a) a TCRα chain that comprises a constant domain comprising minimal murinization amino acid substitutions at positions 90, 91, 92, and 93, and hydrophobic amino acid substitutions at positions 115, 118, and 119;
(b) a polypeptide cleavage signal; and
(c) a TCRβ chain that comprises a constant domain comprising minimal murinization amino acid substitutions at positions 18, 22, 133, 136, and 139,
wherein the fusion protein does not bind MAGEA4.

12. A fusion protein comprising:
(a) a TCRα chain that comprises a constant domain comprising the amino acid substitutions, P90S, E91D, S92V, S93P, S115L, G118V, and F119L;
(b) a polypeptide cleavage signal; and
(c) a TCRβ chain that comprises a constant domain comprising the amino acid substitutions E18K, S22A, F133I, E/V136A, and Q139H,
wherein the fusion protein does not bind MAGEA4.

13. A fusion protein comprising:
(a) a TCRα chain that comprises a constant domain comprising at least 4 minimal murinization amino acid substitutions and at least 3 hydrophobic amino acid substitutions in the TCRα chain transmembrane domain, wherein the TCRα chain constant domain comprises an amino acid sequence that is at least 95%, 96%, 97%, 98%, or 99% identical to the amino acid sequence set forth in SEQ ID NO: 4;
(b) a polypeptide cleavage signal; and
(c) a TCRβ chain that comprises a constant domain comprising at least 5 minimal murinization amino acid substitutions, wherein the TCRβ chain constant domain comprises an amino acid sequence that is at least 95%, 96%, 97%, 98%, or 99% identical to the amino acid sequence set forth in SEQ ID NO: 5 or SEQ ID NO: 6,
wherein the fusion protein does not bind MAGEA4.

14. A fusion protein comprising:
(a) a TCRα chain comprising a constant domain comprising the amino acid sequence set forth in SEQ ID NO: 4;
(b) a polypeptide cleavage signal; and
(c) a TCRβ chain comprising a constant domain comprising the amino acid sequence set forth in SEQ ID NO: 5 or SEQ ID NO: 6,
wherein the fusion protein does not bind MAGEA4.

15. The fusion polypeptide of any one of claims 9 to 14, wherein the polypeptide cleavage signal is a viral self-cleaving peptide or ribosomal skipping sequence.

16. The fusion polypeptide of any one of claims 9 to 15, wherein the polypeptide cleavage signal is a viral 2A peptide.

17. The fusion polypeptide of any one of claims 9 to 16, wherein the polypeptide cleavage signal is an aphthovirus 2A peptide, a potyvirus 2A peptide, or a cardiovirus 2A peptide.

18. The fusion polypeptide of any one of claims 9 to 17, wherein the polypeptide cleavage signal is a viral 2A peptide selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

19. A nucleic acid encoding the TCR according to any one of claims 1 to 8 or the fusion protein of any one of claims 9 to 18.

20. A nucleic acid comprising a first polynucleotide encoding a minimally murinized TCRα chain wherein the TCRα chain transmembrane domain comprises hydrophobic amino acid substitutions; an internal ribosomal entry site (IRES); and a second polynucleotide encoding a minimally murinized TCRβ chain, wherein the fusion protein does not bind MAGEA4.

21. A nucleic acid comprising:
(a) a first polynucleotide encoding a TCRα chain that comprises a constant domain comprising minimal murinization amino acid substitutions at positions 90, 91, 92, and 93, and hydrophobic amino acid substitutions at positions 115, 118, and 119;
(b) an IRES; and
(c) a second polynucleotide encoding a TCRβ chain that comprises a constant domain comprising minimal murinization amino acid substitutions at positions 18, 22, 133, 136, and 139,
wherein the fusion protein does not bind MAGEA4.

22. A nucleic acid comprising:
(a) a first polynucleotide encoding a TCRα chain that comprises a constant domain comprising the amino acid substitutions, P90S, E91D, S92V, S93P, S115L, G118V, and F119L;
(b) an IRES; and
(c) a second polynucleotide encoding a TCRβ chain that comprises a constant domain comprising the amino acid substitutions E18K, S22A, F133I, E/V136A, and Q139H,
wherein the fusion protein does not bind MAGEA4.

23. A nucleic acid comprising:
(a) a first polynucleotide encoding a TCRα chain that comprises a constant domain comprising at least 4 minimal murinization amino acid substitutions and at least 3 hydrophobic amino acid substitutions in the TCRα chain transmembrane domain, wherein the TCRα chain constant domain comprises an amino acid sequence that is at least 95%, 96%, 97%, 98%, or 99% identical to the amino acid sequence set forth in SEQ ID NO: 4;
(b) an IRES; and
(c) a second polynucleotide encoding a TCRβ chain that comprises a constant domain comprising at least 5 minimal murinization amino acid substitutions, wherein the TCRβ chain constant domain comprises an amino acid sequence that is at least 95%, 96%, 97%, 98%, or 99% identical to the amino acid sequence set forth in SEQ ID NO: 5 or SEQ ID NO: 6,
wherein the fusion protein does not bind MAGEA4.

24. A nucleic acid comprising:
(a) a first polynucleotide encoding a TCRα chain comprising a constant domain comprising the amino acid sequence set forth in SEQ ID NO: 4;
(b) an IRES; and
(c) a second polynucleotide encoding a TCRβ chain comprising a constant domain comprising the amino acid sequence set forth in SEQ ID NO: 5 or SEQ ID NO: 6,
wherein the fusion protein does not bind MAGEA4.

25. A vector comprising a nucleic acid encoding the TCR of any one of claims 1 to 8 or the fusion protein of any one of claims 9 to 18.

26. A vector comprising the nucleic acid of any one of claims 19 to 24, wherein the vector is preferably an expression vector, more preferably a retroviral vector or even more preferably a lentiviral vector.

27. A cell expressing the TCR of any one of claims 1 to 8.

28. A cell expressing the fusion protein of any one of claims 9 to 18.

29. A cell comprising the nucleic acid of any one of claims 19 to 24.

30. A cell comprising the vector of claim 25 or claim 26.

31. The cell of any one of claims 27 to 30, wherein the cell is an immune effector cell.

32. The cell of any one of claims 27 to 31, wherein the cell is an immune effector cell selected from the group consisting of: a T cell, a natural killer (NK) cell, or a natural killer T (NKT) cell.

33. A composition comprising the TCR of any one of claims 1 to 8, the fusion protein of any one of claims 9 to 18, the nucleic acid of any one of claims 19 to 24, the vector of claim 25 or claim 26, or the cell of any one of claims 27 to 32.

34. A pharmaceutical composition comprising a pharmaceutically acceptable carrier and the TCR of any one of claims 1 to 8, the fusion protein of any one of claims 9 to 18, the nucleic acid of any one of claims 19 to 24, the vector of claim 25 or claim 26, or the cell of any one of claims 27 to 32.

35. The TCR of any one of claims 1 to 8, the fusion protein of any one of claims 9 to 18, the nucleic acid of any one of claims 19 to 24, the vector of claim 25 or claim 26, or the cell of any one of claims 27 to 32, the composition of claim 33, or the pharmaceutical composition of claim 34 for use as a medicament.

36. The TCR of any one of claims 1 to 8, the fusion protein of any one of claims 9 to 18, the nucleic acid of any one of claims 19 to 24, the vector of claim 25 or claim 26, or the cell of any one of claims 27 to 32, the composition of claim 33, or the pharmaceutical composition of claim 34 for use in the treatment of cancer, wherein the cancer is preferably a hematological cancer or a solid tumor, more preferably wherein the cancer is selected from the group consisting of sarcoma, prostate cancer, uterine cancer, thyroid cancer, testicular cancer, renal cancer, pancreatic cancer, ovarian cancer, esophageal cancer, non-small-cell lung cancer, non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia, most preferably wherein the cancer is selected from the group consisting of NSCLC, SCLC, breast, ovarian or colorectal cancer, sarcoma or osteosarcoma.
